(19) United States
(12) Patent Application Publication
(10) Pub. No.: US 2017/0255755 A1
(43) Pub. Date: Sep. 7, 2017

(54) DECISION SUPPORT DEFINITION EDITOR

(71) Applicant: Physio-Control, Inc., Redmond, WA (US)

(72) Inventors: Kenneth J. PETERSON, Bellevue, WA (US); John DAYNES, Redmond, WA (US); Mitchell Smith, Sammamish, WA (US); Jeffery Edwards, Bellingham, WA (US); David B. Stewart, Carnation, WA (US); Cheryl Protas, Redmond, WA (US)

(21) Appl. No.: 15/518,439

(22) PCT Filed: Oct. 12, 2015
(86) PCT No.: PCT/US2015/055095

§ 371 Date: Apr. 11, 2017

Related U.S. Application Data
(60) Provisional application No. 62/063,744, filed on Oct. 14, 2014.

(51) Int. Cl. G06F 19/00; G06N 5/04

(52) U.S. Cl. CPC G06F 19/3418; G06F 19/345; G06F 19/3412; G06N 5/043; A61N 1/39

(57) ABSTRACT

A decision support definitions library stores one or more decision support definitions for providing appropriate treatment paths. A device management library stores device and configuration setting information on one or more devices associated with an organization, the configuration setting including information on smart agents residing on the one or more devices. A decision support definition editor is configured to modify a selected one or more decision support definitions and one or more device and configuration settings. A decision support definition generator is configured to create an installation configuration file including an entry for each of a selected one or more decision support definitions, wherein each entry comprises: (i) information that identifies the respective decision support definition, (ii) configuration information, and (iii) information that identifies an installation program. A deployment manager is configured to distribute the installation configuration file and the smart agent with selected decision support definitions.

### RELATED APPLICATION

This application claims the benefit of U.S. Prov. Appl. 62/063,744, filed 14 Oct. 2014, of the same title, and which is incorporated by reference in its entirety.

### FIELD

This disclosure generally relates to decision support architectures for medical devices.

### BACKGROUND

A defibrillator is one medical device that is used to treat medical conditions. There are other medical devices including those recognized by the U.S. Food and Drug Administration for use in diagnosing, preventing, or treating disease or other conditions.

Advances in medical devices have included provisioning the medical devices with hardware and software for generating alerts that provide coaching to a caregiver on a medical treatment. For example, a medical device may issue instruction events, and even prompts, for the caregiver to perform CPR more effectively. Advanced medical devices may also provide a caregiver with advanced coaching instructions on specific procedures to follow for treating a patient for various conditions such as sepsis, infection, bacteremia, SIRS, trauma, burns, pancreatitis, etc. Many advanced medical devices employ decision trees for navigating a caregiver through the many and varied instructions that may make up a protocol for the treatment of a particular condition of the patient. Caregivers may benefit from enhancements to these and other decision support instructions and alerts that may make the coaching of a caregiver provided by the medical device more effective, efficient, and strategic.

### BRIEF SUMMARY

This disclosure is directed generally to providing intelligent agents for a decision support system thereby enhancing analysis through linking and sharing information using knowledge and experience distributed among intelligent agents and caregivers.

A computer-implemented system and method are described for providing a configurable frame work for creation and distribution of smart agents. More specifically, a decision support definitions library is configured to store one or more decision support definitions for providing appropriate treatment paths. A device management library is configured to store device and configuration setting information on one or more devices associated with an organization, the configuration setting including information on smart agents residing on the one or more devices. A decision support definition editor is configured to modify a selected one or more decision support definitions and one or more device and configuration settings. A decision support definition generator is configured to create an installation configuration file including an entry for each of a selected one or more decision support definitions taken from the decision support definitions library, wherein each entry for the selected one or more decision support definitions comprises: (i) information that identifies the respective decision support definition, (ii) configuration information on one or more devices associated with an organization taken from the device management library to be provided the installation configuration file, and (iii) information that identifies an installation program to use in installing the respective decision support definitions on one or more devices associated with an organization to be provided the installation configuration file. A deployment manager is configured to distribute the installation configuration file and the smart agent with selected decision support definitions.

These and other features and advantages of this description will become more readily apparent from the following Detailed Description, which proceeds with reference to the drawings, in which:

### BRIEF DESCRIPTION OF THE DRAWINGS

FIG. 1 is an illustrative diagram of a scene showing the use of an external defibrillator to save the life of a person according to this disclosure.

FIG. 2 is a table listing two illustrative types of the external defibrillator shown in FIG. 1, and who they might be used by.

FIG. 3 is a diagram showing components of an external defibrillator, such as the one shown in FIG. 1, configured in an illustrative embodiment according to this disclosure.

FIG. 4A depicts an illustrative computer-implemented system for providing a configurable frame work for creation and distribution of smart agents according to this disclosure.

FIG. 4B depicts an illustrative decision support definition editor module depicted in FIG. 4A for creation and distribution of smart agents according to this disclosure.

FIGS. 4C and 4D show illustrative templates that may be included in a template library of this disclosure.

FIG. 5 depicts an illustrative embodiment of the decision support definition editor of the decision support definition editor module shown in FIG. 4B.

FIGS. 6A, 6B, and 7 show illustrative functional diagrams of illustrative systems, each defining a medical enterprise, for distributing the installation configuration file and the smart agent with selected decision support definitions to medical devices according to this disclosure.

FIG. 8 depicts an illustrative embodiment of a computer-implemented method for providing a configurable frame work for creation and distribution of smart agents according to this disclosure.

FIG. 9 depicts an illustrative process for selecting, editing, and deploying a new smart agent according to this disclosure.

FIG. 10 depicts an illustrative process for selecting, editing, and deploying an existing smart agent according to this disclosure.

### DETAILED DESCRIPTION

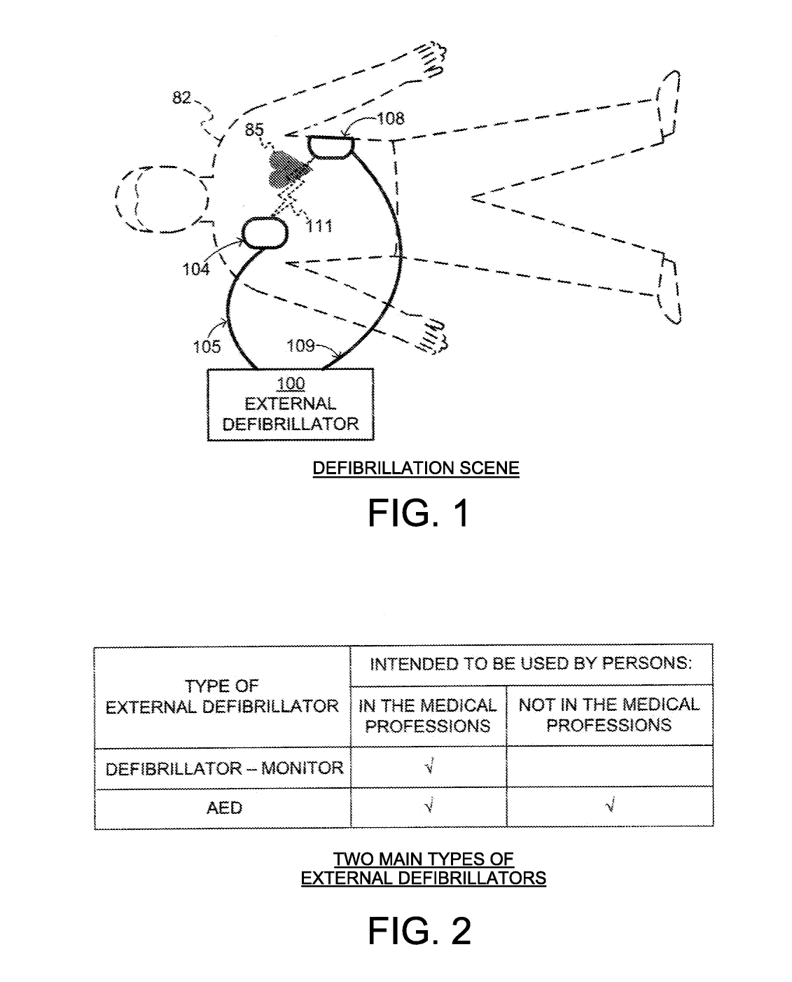

FIG. 1 is a diagram of a defibrillation scene showing the use of an external defibrillator to save the life of a person according to this disclosure. As shown, a person 82 is lying on his back. Person 82 could be a patient in a hospital, or someone found unconscious, and then turned over onto his back. Person 82 is experiencing a condition in their heart 85, which could be Ventricular Fibrillation (VF).

A portable external defibrillator 100 has been brought close to person 82. At least two defibrillation electrodes 104, 108 are typically provided with external defibrillator 100, and are sometimes called electrodes 104, 108. Electrodes 104, 108 are coupled together with external defibrillator 100 via respective electrode leads 105, 109. A rescuer (not shown) has attached electrodes 104, 108 to the skin of person 82. Defibrillator 100 is administering, via electrodes 104, 108, a brief, strong electric pulse 111 through the body of person 82. Pulse 111, also known as a defibrillation shock, also goes through heart 85, in an attempt to restart it, for saving the life of person 82.

Defibrillator 100 can be one of different types, each with different sets of features and capabilities. The set of capabilities of defibrillator 100 is determined based upon who would use it and what training they would be likely to have. Examples are now described.

FIG. 2 is a table listing two typical types of external defibrillators, and who they are primarily intended to be used by. A first type of defibrillator 100 is generally called a defibrillator-monitor, because the defibrillator part is typically formed as a single unit with a patient monitor part. A defibrillator-monitor is sometimes called monitor-defibrillator. A defibrillator-monitor is intended to be used by persons in the medical profession, such as doctors, nurses, paramedics, emergency medical technicians, etc. who may be trained to provide medical treatment to the patient during a defibrillation process based upon information provided by the monitor. Such a defibrillator-monitor is intended to be used in a pre-hospital or hospital scenario.

The defibrillator part may be dedicated to a particular mode of operation. Alternatively, the defibrillator part may be configured to operate in more than one modes of operation. One mode of operation of the defibrillator part may be that of an automated defibrillator, which can determine whether a shock is needed and, if so, charge to a predetermined energy level and instruct the user to administer the shock. Another mode of operation may be that of a manual defibrillator, where the user determines the need and controls administering the shock. In this embodiment, one illustrative defibrillator is configured to enable both automated defibrillation and manual defibrillation modes of operation depending upon the selection of the user. As a patient monitor, the device has features additional to what is minimally needed for mere operation as a defibrillator. These features can be for monitoring physiological indicators of a person in an emergency scenario. These physiological indicators are typically monitored as signals. For example, these signals can include a person's full ECG (electrocardiogram) signals, or impedance between two electrodes. Additionally, these signals can be about the person's temperature, non-invasive blood pressure (NIBP), arterial oxygen saturation/pulse oximetry (SpO2), the concentration or partial pressure of carbon dioxide in the respiratory gases, which is also known as capnography, and so on. These signals can be further stored and/or transmitted as patient data.

A second type of external defibrillator 100 is generally called an AED, which stands for “Automated External Defibrillator”. An AED typically makes the shock/no shock determination by itself, automatically. Indeed, it can sense enough physiological conditions of the person 82 via only the shown defibrillation electrodes 104, 108 of FIG. 1. In its present embodiments, an AED can either administer the shock automatically, or instruct the user to do so, e.g. by pushing a button. Being of a much simpler construction, an AED typically costs much less than a defibrillator-monitor. As such, it makes sense for a hospital, for example, to deploy AEDs at its various floors, in case the more expensive defibrillator-monitor is more critically being deployed at an Intensive Care Unit, and so on.

AEDs, however, can also be used by people who are not trained in the medical profession. More particularly, an AED can be used by many professional first responders, such as policemen, firemen, etc. Even a person with only first-aid training can use one. And AEDs increasingly can supply instruction events to whoever is using them.

AEDs are thus particularly useful, because it is so critical to respond quickly, when a person suffers from VF. Often, the people who will first reach the VF sufferer may not be in the medical profession.

Increasing awareness of the short survival time of a patient experiencing a VF, has resulted in AEDs being deployed more pervasively in public or semi-public spaces, enabling members of the public to use one provided they have obtained first aid and CPR/AED training. In this way, defibrillation can be administered sooner after the onset of VF, to hopefully be effective in rescuing the person.

There are additional types of external defibrillators, which are not listed in FIG. 2. For example, a hybrid defibrillator can have aspects of an AED, and also of a defibrillator-monitor. An illustrative example may be an AED provided with an ECG monitoring capability.

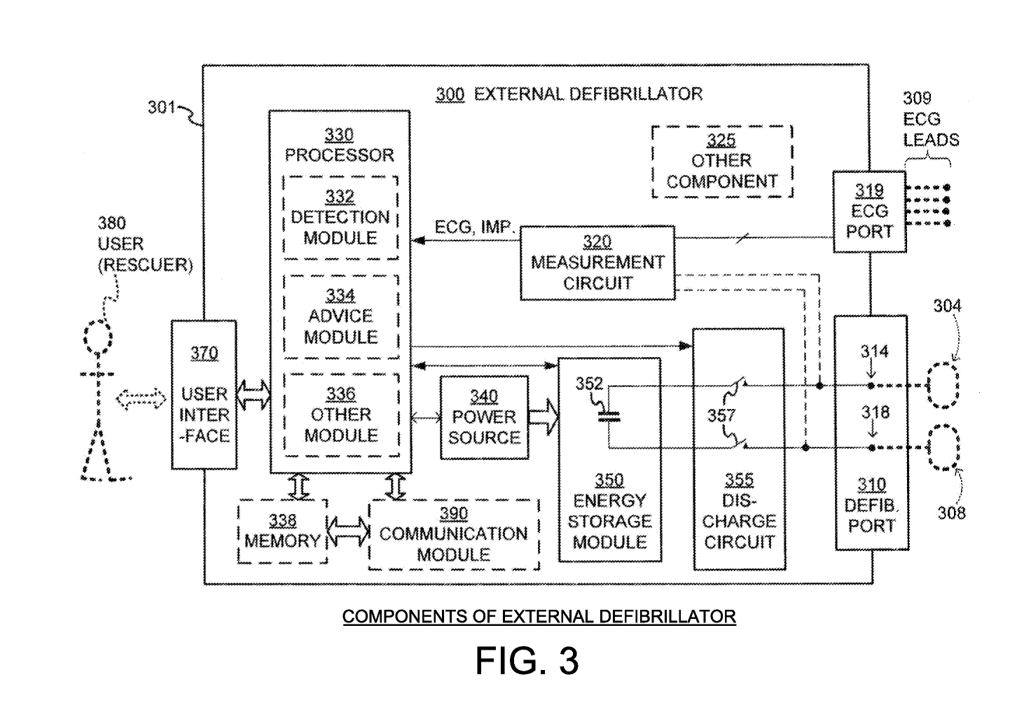

FIG. 3 is a diagram showing components of an external defibrillator 300 configured in an illustrative embodiment according to this disclosure. These components can be configured, for example, in external defibrillator 100 of FIG. 1. Plus, these components of FIG. 3 can be provided in a housing 301, which is also known as casing 301.

External defibrillator 300 is intended for use by a user 380, who would be the rescuer. Defibrillator 300 typically includes a defibrillation port 310, which may be configured as a socket (not shown) in housing 301. Defibrillation port 310 includes nodes 314, 318. Defibrillation electrodes 304, 308, which can be similar to electrodes 104, 108 in FIG. 1, can be plugged into defibrillation port 310, so as to make electrical contact with nodes 314, 318, respectively. It is also possible that electrodes can be hard-wired to defibrillation port 310, etc. Either way, defibrillation port 310 can be used for guiding to person 82 via electrodes an electrical charge that has been stored in defibrillator 300, as discussed below.

If defibrillator 300 is actually a defibrillator-monitor, as was described with reference to FIG. 2, then it will typically also have an ECG port 319 in housing 301, for plugging in ECG leads 309. ECG leads 309 can help sense an ECG signal, e.g. a 12-lead signal, or a signal taken from a different number of leads. Moreover, a defibrillator-monitor could have additional ports (not shown), and another component 325 for the above described additional features, such as for receipt of patient signals.

Defibrillator 300 also includes a measurement circuit 320. Measurement circuit 320 receives physiological signals from ECG port 319, and also from other ports, if provided. These physiological signals are sensed, and information about them is rendered by circuit 320 as data, or other signals, etc.

If defibrillator 300 is actually an AED, it may lack ECG port 319. Measurement circuit 320 can obtain physiological signals in this case through nodes 314, 318 instead, when defibrillation electrodes 304, 308 are attached to person 82. In these cases, a person's ECG signal can be sensed as a voltage difference between electrodes 304, 308. Plus, impedance between electrodes 304, 308 can be sensed for detecting, among other things, whether these electrodes 304, 308 have been inadvertently disconnected from the person.

Defibrillator 300 also includes a processor 330. Processor 330 may be implemented in any number of ways. Such ways include, by way of example and not of limitation, digital and/or analog processors such as microprocessors and digital-signal processors (DSPs); controllers such as microcontrollers; software running in a machine; programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

Processor 330 may include a number of modules. One such module can be a detection module 332, which senses outputs of measurement circuit 320. Detection module 332 can include a VF detector. Thus, the person's sensed ECG can be used to determine whether the person is experiencing VF.

Another such module in processor 330 can be an advice module 334, which arrives at a piece of instructional advice based on outputs of detection module 332. Advice module 334 can include a Shock Advisory Algorithm residing in a memory unit (not shown) in the advice module for instructing the processor to implement decision rules, etc. Alternatively, the Shock Advisory Algorithm may reside in part or in whole on a memory 338 of the defibrillator. The instruction to the processor can be to shock, to not shock, to administer other forms of therapy, and so on. If the instruction to the processor is to shock, in some external defibrillator embodiments, the processor is configured to report that instruction to the user via user interface 370, and to prompt the user to do it. In other embodiments, the processor may be configured to execute the instructional advice, by administering the shock. If the instructional advice is to administer CPR, the processor may be configured to enable defibrillator 300 to issue prompts to administer CPR, etc.

Processor 330 can include additional modules, such as module 336, for other functions. In addition, if other component 325 is provided, it may be operated in part by processor 330 or by another processor.

Defibrillator 300 optionally further includes the memory 338, which can work together with processor 330. Memory 338 may be implemented in any number of ways. Such ways include, by way of example and not of limitation, nonvolatile memories (NVM), read-only memories (ROM), random access memories (RAM), any combination of these, etc. Memory 338, if provided, may include programs containing instruction events for execution by processor 330 or other processors that may be included in the external defibrillator. The programs provide instruction events for execution by the processor 330, and can also include instruction events regarding protocols and decision making analytics, etc. that can be used by advice module 334. In addition, memory 338 can store prompts for user 380, etc. Moreover, memory 338 can store patient data.

Defibrillator 300 may also include a power source 340. To enable portability of defibrillator 300, power source 340 typically includes a battery. Such a battery is typically implemented as a battery pack, which can be rechargeable or not. Sometimes, a combination is used, of rechargeable and non-rechargeable battery packs. Other embodiments of power source 340 can include an AC power override, whereby AC power, instead of power from power source 340 is delivered to an energy storage module 350 when AC power is available. In some embodiments, power source 340 is controlled by processor 330.

Defibrillator 300 additionally includes the energy storage module 350. Module 350 is where electrical energy is stored in preparation for a sudden discharge to administer a shock. The charge to module 350 from power source 340 to the right amount of energy can be controlled by processor 330. In typical implementations, module 350 includes one or more capacitors 352, and may include other circuitry.

Defibrillator 300 moreover includes a discharge circuit 355. Circuit 355 can be controlled to permit the energy stored in module 350 to be discharged to nodes 314, 318, and thus also to defibrillation electrodes 304, 308. Circuit 355 can include one or more switches 357. Those can be made in a number of ways, such as by an H-bridge, and in other ways well known in the art.

Defibrillator 300 further includes the user interface 370 for user 380. User interface 370 can be made in any number of ways. For example, interface 370 may include a screen, to display a parameter of a patient that is detected and measured, provide visual feedback to the rescuer for their resuscitation attempts, and so on. Interface 370 may also include a speaker, to issue voice prompts, etc. Interface 370 may additionally include various controls, such as pushbuttons, keyboards, and so on. In addition, discharge circuit 355 can be controlled by processor 330, or directly by user 380 via user interface 370, and so on.

Defibrillator 300 can optionally include other components. For example, a communication module 390 may be provided for communicating with other devices. Such communication can be performed wirelessly, or via wire, or by infrared communication, and so on. In this way, data can be communicated from the defibrillator 300 to external devices, such as patient data, incident information, therapy attempted, CPR performance, and so on.

The defibrillator device just described provides one illustrative medical device that may be used with this disclosure. There are other medical devices that may be used with this disclosure including those recognized by the U.S. Food and Drug Administration for use in diagnosing, preventing, or treating disease or other conditions.

In the treatment of a patient, information of a patient, such as conditions of the patient, temperature, pulse rate, visible signs of injury, trauma, etc., or other conditions of the body, may be observed or detected by the caregiver. The caregiver may use that information to take some corrective measure such as administering a treatment to the patient. The feedback response of the patient to the treatment is observed or detected by the caregiver and the caregiver may respond to this feedback by taking further corrective measures. This process of making corrective measures may continue until a point is reached where further corrective measures are no longer required.

In more sophisticated treatment system, a medical device, such as a defibrillator described above and which may be instrumented with ECG or other instrumentation may be used to provide a medical treatment. The medical device may obtain patient parameter data which may include one or more of the following measurements: a measurement of CO₂ exhaled by a patient; an electrical activity of the heart of a patient; an exchange of air between the lungs of a patient and the atmosphere; a pressure of the blood in a patient; a temperature of a patient; an oxygen saturation in the blood of a patient; a chest compression of a patient; an image of the internal structure of a patient; an oxygen saturation in the blood in the brain of a patient; the acidity or alkalinity of fluids in a patient; or other patient parameter.

The patient parameter of the CO₂ exhaled by a patient may be measured using capnography techniques. The patient parameter of the electrical activity of the heart of a patient may be measured using ECG techniques. The patient parameter of the exchange of air between the lungs of a patient and the atmosphere may be measured using ventilation techniques. The patient parameter of the measurement of the pressure of the blood in a patient may be measured using non-invasive blood pressure measurement techniques or invasive blood pressure measurement techniques. The patient parameter of the temperature of a patient may be measured using temperature measurement techniques. The patient parameter of the oxygen saturation in the blood of a patient may be measured using pulse oximeter techniques or tissue oximetry techniques. The patient parameter of the chest compression of a patient may be measured using chest compression detection and feedback techniques. The patient parameter of the image of the internal structure of a patient may be measured using ultrasound measurement techniques. The patient parameter of the oxygen saturation in the blood in the brain of a patient may be measured using cerebral oximetry techniques. The patient parameter of the acidity or alkalinity of fluids in a patient may be measured using non-invasive pH measurement techniques. These and other techniques and modules for generating the foregoing and other kind of patient parameter data for use with this disclosure are well known in the art. Medical devices may provide a caregiver with certain patient parameter or other data. A caregiver may use this data along with information of a patient that the caregiver may observe or detect, such as conditions of the patient, temperature, pulse rate, visible signs of injury, trauma, etc., or other condition of the body, in performing a treatment on a patient.

Medical devices may also be provided with hardware and software configured to provide a protocol of events that has been programmed into the medical device for the purpose of providing events for coaching the caregiver on a treatment to be administered to the patient. The protocol of events may define a decision tree of events that a caregiver should follow for administering a particular treatment.

In practice, a protocol may be initiated by caregiver selection of some branch in the decision tree. The decision tree then typically navigates through the branches of the decision tree based upon that selection. More specifically, a processor of the medical device executes the protocol to create a processing thread of instructions that navigates through the decision tree. Based on the tests performed at the nodes of the tree, the processing thread may issue instructions, alerts, etc., to coach the caregiver through a medical treatment. These and other like decision tree constructs provide a decision support system to a caregiver.

Decision support systems have been enhanced in several ways. In some instances, the decision support system may be dedicated to treatment of a particular medical condition in which case the medical device may be provided with more specialized treatment instructions. In others, the decision support system may be configured to interact with a caregiver such as by waiting on input from a caregiver before issuing the next instruction. This allows the decision support system to factor the feedback or other input of a caregiver into the next instructions, alerts, etc. provided by the decision support system. In other cases, the feedback or other input of a caregiver may be used to modify decision rules implemented by the decision support system. These systems automatically select clinical guidelines applicable to the care of a patient and track the progress of the patient through a stage of the guideline.

These and other decision support tools provides help to the clinical user in diagnosing patient conditions, determining appropriate treatment paths, and recording what treatment activities have occurred. Help may be provided through a series of smart algorithms to help identify potential patient conditions, messages/reminders to inform the user to perform some task or action, a series of checklists helping the user follow a pre-defined treatment path, or the display of reference material or calculators to help the user determine proper course of action or dosing.

Having thus introduced background on the general operation of decision support systems for use with medical devices, we now turn to features that are provided by this disclosure.

In order for decision support tools to be desired and useful, they should be customizable/tailored to the specific patient populations, treatment protocols, and care paths for a specific provider. There are standards of care for some disease states that are shared at a local or international level, and other that are left to caregiver discretion. This disclosure provides an editor that allows the Medical Director to download and edit decision support tools that others have already created and let them share their specific decision support definitions with other users.

More specifically, in accordance with this disclosure, a decision support definitions library stores one or more decision support definitions for providing appropriate treatment paths. A device management library stores device and configuration setting information on one or more devices associated with an organization, the configuration setting including information on smart agents residing on the one or more devices. A decision support definition editor is configured to modify a selected one or more decision support definitions and one or more device and configuration settings. A decision support definition generator is configured to create an installation configuration file including an entry for each of a selected one or more decision support definitions, wherein each entry comprises: (i) information that identifies the respective decision support definition, (ii) configuration information, and (iii) information that identifies an installation program. A deployment manager is configured to distribute the installation configuration file and the smart agent with selected decision support definitions.

The following decision support definition terminology taxonomy is provided in support of this disclosure.

Decision Support Definitions are what a customer creates using a Decision Support Definition Editor, as defined below, to have all kinds of customer programmable (“Customer Programmable”) help objects such as checklists, messages, reference materials, smart algorithms (“Smart Algorithms”), drug calculators (“Drug Calculators”), etc. displayed on the user interface of a customer's medical device(s) (“Customer's Medical Devices”) and Medical Device Software Applications, defined below, when a predefined set of inputs (“Entrance Criteria”) is received. The Decision Support Definitions are made up of Decision Support Definition Sets and Software Managers, as defined below.

The Decision Support Definition Sets are what the Decision Support Definition Editor creates and loads into a user's medical device(s) (“User's Medical Devices”) to work in conjunction with Software Managers contained within those devices to create the functionality of the Decision Support Definitions. These Decision Support Definition Sets include the following types which are defined below: Intelligent Agents, Presentation Definitions, Output Definitions, Reference Material, and Smart Algorithms.

Intelligent Agents encapsulate an entrance criteria (“Entrance Criteria”) definition and execute concurrently with the Software Managers that control the presentation to the users.

Presentation Definitions inform the Software Managers how to display customized user information based on pre-defined presentation tablets (“Presentation Templates”).

Output Definitions inform the Software Managers what output messages to create when the user selects each input on a user interface (“User Interface”) defined by the Presentation Definitions.

Reference Material is information (text and graphics) that can be displayed on the User Interface of one of the devices in the disclosed system. The Decision Support Definition Editor can decide when Reference Material is displayed on a device in the system.

Smart Algorithms are individual software algorithms that calculate a new output based on a pre-defined set of patient data inputs. Examples of Smart Algorithms include Smart Vital Indices, Drug Dosage Calculators, and Count-down Timers. The Decision Support Definition Editor can decide when Smart Algorithms are initiated on a device in the system. The output can be used as entrance criteria for the Intelligent Agents.

The Software Managers that make up the Decision Support Definitions are software processes that independently operate within the Medical Device to perform some type of transformation. There could be one or many Software Managers operating within the Medical Device.

External System Elements are the systems that interface with the Decision Support Definition Editor to successfully implement the Decision Support Definitions. The External System Elements include the following elements which are defined below: Decision Support Definition Library, Device Definition Library, Web (World Wide Web), Monitor or Monitor/Defibrillator, and Medical Device Software Applications.

The Decision Support Definition Library is a collection of Decision Support Definitions created and used by the customers of the system. Some of these are designated as shared and available to be review and edited by all customers of the System. The Library can be hosted on a central server (“Central Server”) or distributed across multiple storage mechanisms owned by the various customers.

The Device Definition Library contains the software and configuration data of the customer devices. The library can exist on a central server or locally managed on customer device. They capture the data as registered and updated version of each device owned by customers. They verify the configuration information is correct before updating to the latest set of Decision Support Definitions by uploading the Decision Support Definition Software Sets from the Decision Support Definition Editor

The Web (World Wide Web) is a Repository of shared material (“Shared Material”) external to the Medical Device, such as for reference material (“Reference Material”) that could be edited and downloaded in the Customer owned devices.

The Monitor or Monitor/Defibrillator is one class of Medical Device that Decision Support Definitions can be deployed on to provide the Customer's programmable help objects. The Software Managers running on the Monitor use the Decision Support Definition Software Sets to determine how to implement the Decision Support Definitions.

Medical Device Software Applications are Medical device software that runs on hardware (“HW”) such as Tablet or Laptop computers, Smart Phones, or other Mobile Devices that contain Software Managers that use the Decision Support Definitions to provide the Customer's programmable help objects.

The Decision Support Definition Editor is what the Customer uses to creates the Decision Support Definitions that are used by the Software Managers on the customer's devices. It is made up of the following functions (these could be implemented in a variety of ways) defined below: a Directory Viewer and an Editor.

The Directory Viewer is used to create new or view and manage the previously generated Decision Support Definitions that are stored on the Decision Support Definition Library.

The Editor modifies the Decision Support Definitions. It is made up of the following functions. These could be implemented in a variety of ways including a Version Control (check-in/check-out), an Entrance Criteria (Intelligent Agent), and a display generator (“Display Generator”) (“Display Manager”).

The Version Control (check-in/check-out) assigns the name and version number to the Decision Support Definitions that are being edited and what device type and group the Decision Support Definitions are allocated to.

The Entrance Criteria (Intelligent Agent) defines the entrance criteria necessary to trigger the Decision Support Definitions.

The Display Generator (Display Manager) uses the user Interface templates in the Template Library to define the User Interface Presentation of the Decision Support Definition on the associated device type.

The Result Generator (Response Manager) determines the outputs generated from each user interface control (“User Interface Control”) on the Display Generator.

The Smart Algorithms determine the transformation of available inputs to outputs. The customer has the ability to edit what a smart algorithm produces based on available inputs

Device Definition Sets Manager provides an interface into the Device Definition Library to identify all devices that have been registered by the customer and place those devices into groups for application of the Decision Support Definitions. It also allows updates to the Device Definition Data to be uploaded to the Device Definition Library

Template Library provides the pre-defined user interface templates (“User Interface Templates”) that are already supported by the Software Managers that operate on the various devices that make up the System.

Decision Support Definition Set Generator uses the inputs entered by the editor to create the Decision Support Software Definition Sets that are loaded into the customer's devices via the Deployment Manager. Includes consistency checking that performs a verification that all outputs and inputs are consistent across the Decision Support Definitions and Device Configuration Data that are defined for this device in the Device Definition Library.

Presentation Simulator provides a simulation of the Medical Devices user interface onto the Editors Platform that will appear on the various devices within the customers system. Customer may be able to simulate all of the inputs, defined in the Decision Support Definitions and visualize the response to those inputs created by the Decision Support Definitions.

Deployment Manager provides the capability of the customer to manage approvals/authorization/authentication of the Decision Support Definitions and works in conjunction with the Device Definition Library to authorize and track the deployment of new Decision Support Software Definition Sets.

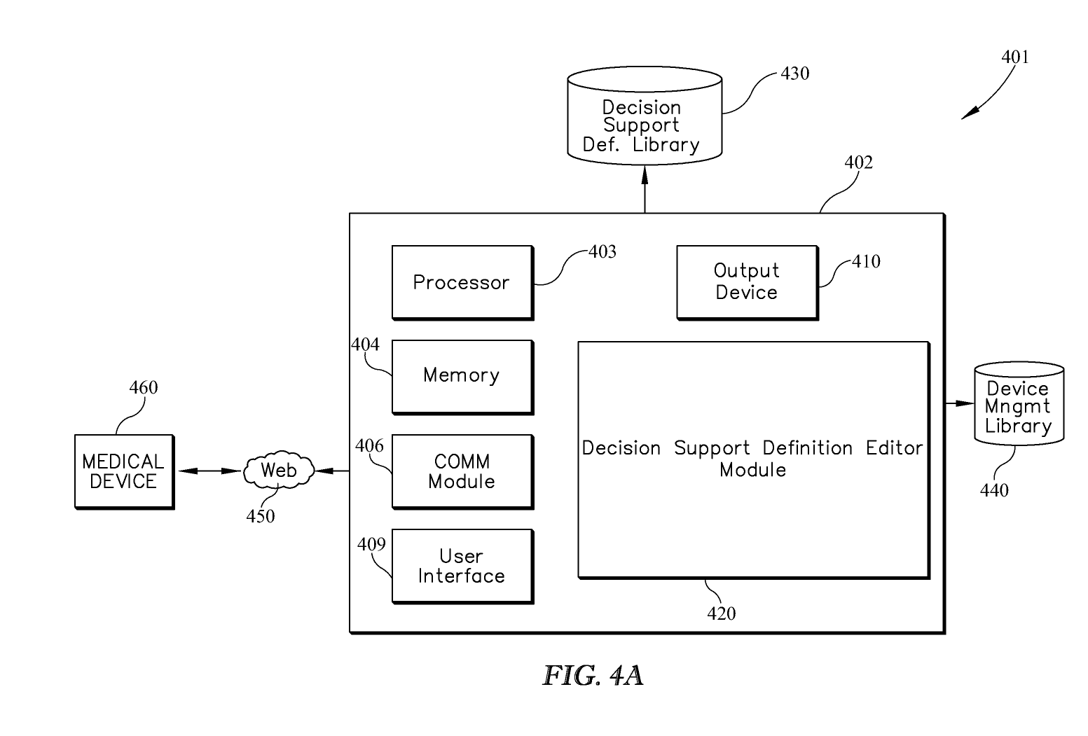

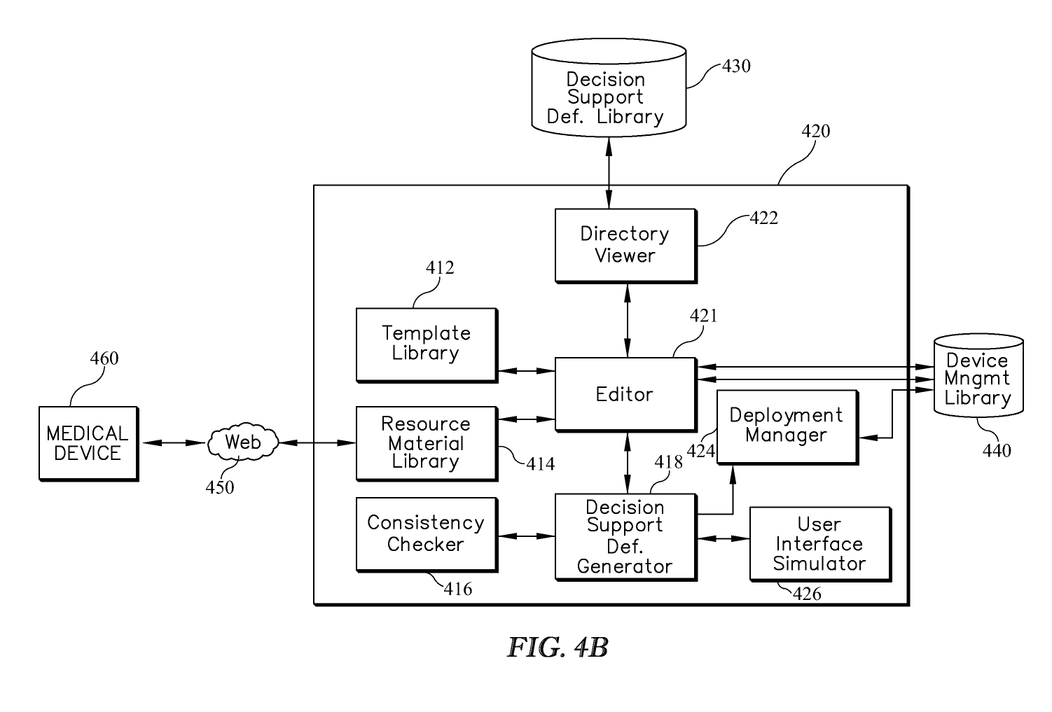

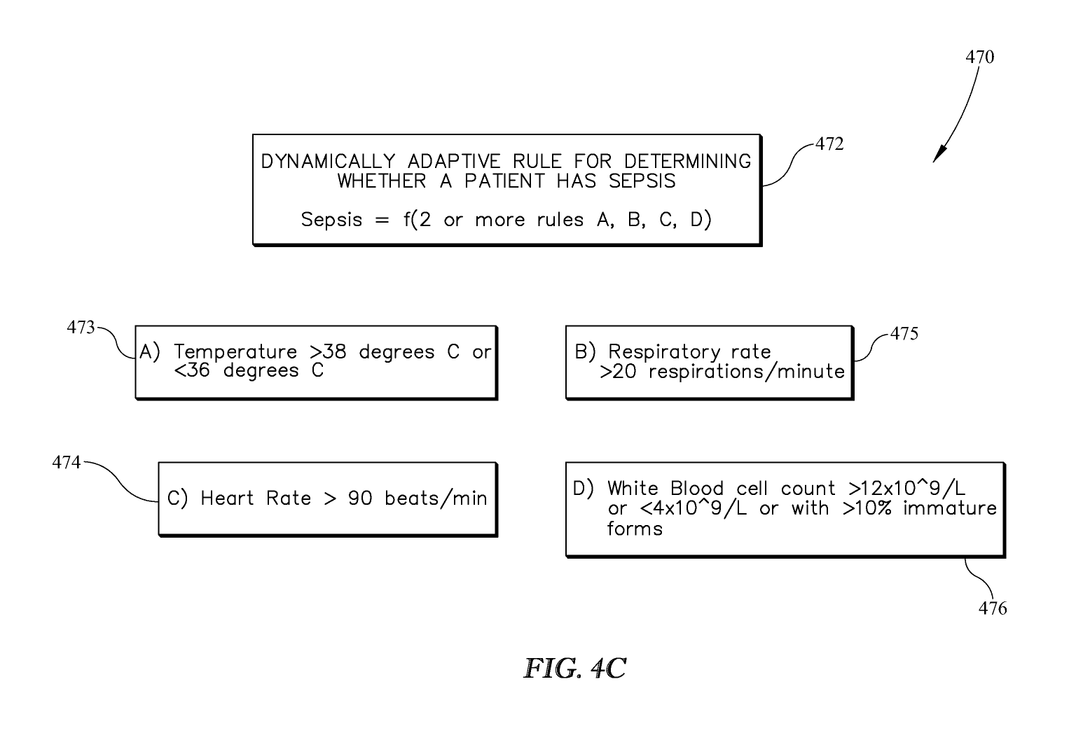

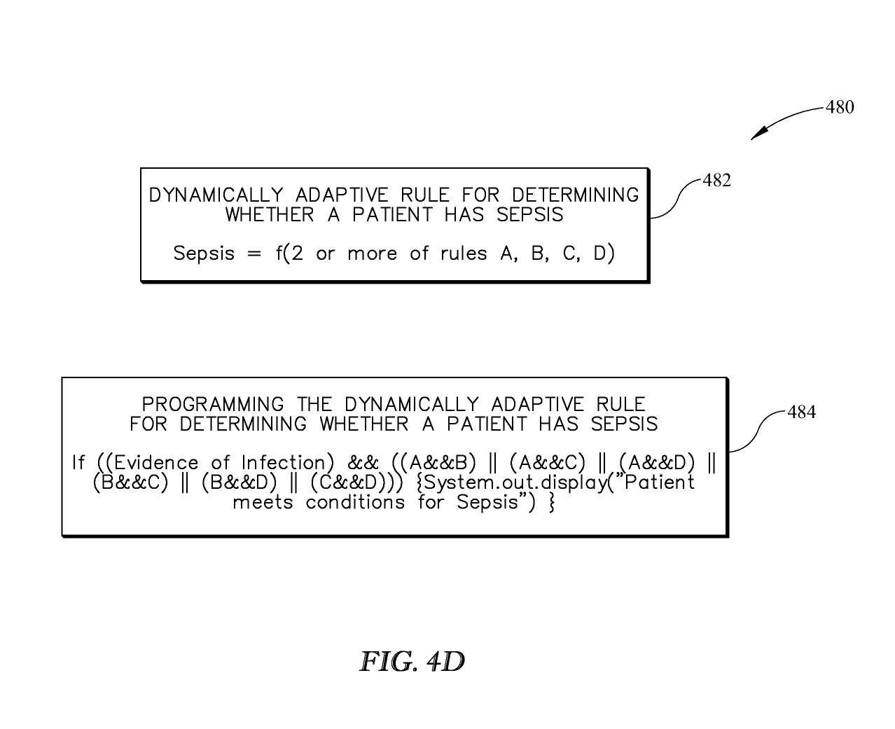

Turning now to FIG. 4A, FIG. 4A provides an illustrative computer system 401 according to this disclosure that provides a configurable frame work for creation and distribution of smart agents. The computer system 401 comprises a computing device 402, a decision support definitions library 430, a device management library 440, a network 450, and one or more medical devices 460.

The computing device 410 illustratively includes a processor 403, a memory 404, a communication module 406, a user interface 409, and an output device 410.

The processor 403 may be implemented in any number of ways. Such ways include, by way of example and not of limitation, digital and/or analog processors such as microprocessors and digital-signal processors (DSPs); controllers such as microcontrollers; software running in a machine; programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

The memory 404 are computer-readable storage media that may be encoded with computer-executable instructions (e.g., software) that implement or enable the system. Memory can be any form of data storage. It may be at least one of random access memory (RAM) and/or read only memory (ROM). Information can be stored permanently until overwritten and/or stored temporarily for use while the unit is actively. One or more storage devices (not shown) may also be used to store information in the illustrative computer system 401. The storage devices may illustratively include disk drives or other non-volatile storage media.

The communication module 406 is hardware and software configured to provide for communicating with other devices. Such communication can be performed wirelessly, or via wire, or by infrared communication, and so on. In this way, data structures and message structures may be transmitted via a data transmission medium, such as a signal on a communication link from the computing device to medical devices 460 on the network.

In an illustrative embodiment, the communication module may comprise a wireless module for establishing a wireless communication link with a network. The wireless module may illustratively be a Wi-Fi module. Alternatively, the wireless module may be a blue tooth module, a CDMA module, or any other communication module that enables a wireless communication link for the bidirectional flow of data between devices wirelessly. In alternative embodiments, the communication module establishes a wired communication link with a network via a USB connector, an RS232 connector, or other hardwire connectors well known in the art.

The communication module may use various communication links, such as the Internet, a local area network, a wide area network, a point-to-point dial-up connection, a cell phone network, and so on.

The user interface 409 may be implemented in any number of ways. For example, interface 409 may include a screen, to display data. Interface 409 may also include a speaker (not shown), to issue voice prompts, etc. Interface 409 may be a touch screen keypad that is rendered on the display and which allows a user to enter data or to read data that is rendered on a display. Interface 409 may additionally include various controls, such as pushbuttons, keyboards, and so on. In addition, discharge circuit 355 can be controlled by processor 403, or directly by a user via user interface 409, and so on.

The output device 410 may be output devices such as a visual display capable of displaying data. Displays for use with this disclosure may include an LCD screen, an e-paper display, or other bi-stable display, a CRT display, a touch screen responsive display, or any other type of visual display. The display may be integrated into computing device 410 or it may be external to and in communication with computing device. Illustrative external devices may be a computer selected from the group consisting of a server, a personal computer, a tablet, a mobile computing device, a video device, an ultrasound device, and a printer.

The computing device may be implemented in various operating environments that include personal computers, server computers, handheld or laptop devices, multiprocessor systems, microprocessor-based systems, programmable consumer electronics, network PCs, minicomputers, mainframe computers, distributed computing environments that include any of the above systems or devices, and so on. The computing device may be cell phones, personal digital assistants, smart phones, personal computers, programmable consumer electronics, and so on.

The decision support definition editor module 420 includes an application that runs on a Personal Computer or Tablet Computer that allows a Medical Director to select, edit, and implement Decision Support Tools across a distributed set of medical devices 460. This application can be native to the operating system it is running on, or implemented through a .net/HTML5 implementation. Illustratively, the Editor is a native application running on a Windows 8.1 platform. The functionality and operation of the Editor 420 is described in greater detail later.

The decision support definitions library 430 comprises a database of patient diagnosis and treatment paths and may contain example decision support definitions used in the computer system 401 or used by other computer systems and assimilated use with the computer system 401 of this disclosure. Illustratively, the decision support definitions library may be organized by the patient diagnosis and treatment paths, decision support definitions, both patient diagnosis and treatment paths and decision support definitions, or in other ways.

The device management library 440 is an application that along with service support tools (not shown) determines and manages what possible outputs can be created. For example, the device management library and supporting tool sets (not shown) maintain the latest device configuration and software sets for all customer owned devices. The device manager library and support tools may work alone or in conjunction to maintain consistent configuration across all customer owned devices. In addition, the device management library may also have the ability to determine how often the decision support definitions are used and what specific user inputs have been captured from each use.

The device management library manages downloads of data and applications to the medical devices 460. Software downloads can be delivered to customer's medical devices virtually through network 450, which may be a cloud based communication system. Alternatively, the downloads can be delivered manually through service support tools.

The network 450 illustratively comprises a wide area network such as the Internet, however the network 450 may also comprise a local area network. Further, the network 450 need not be a land-based network, but instead may comprise a wireless network and/or a hybrid of a land-based network and a wireless network for enhanced communications flexibility.

The one or more medical devices 460 is illustrative one or more defibrillators. Alternatively, the one or more medical devices may be a monitor, a defibrillator with a monitor, or any other medical devices including those recognized by the U.S. Food and Drug Administration for use in diagnosing, preventing, or treating disease or other conditions. The monitor may be a medical device viewer such as a personal computer, table computer, wall monitor, etc. that operate together to create an advance tool set for diagnosing and treating patients who are experiencing an emergent condition. Any medical device or combination of medical devices may be used with this disclosure.

FIG. 4B depicts an illustrative decision support definition editor module 420 depicted in FIG. 4A for creation and distribution of smart agents according to this disclosure. The decision support definition editor module 420 comprises a decision support definition editor 421, a directory viewer 422, a decision support definition generator 418, and a deployment manager 424. The decision support definition editor module further includes a template library 412, a resource material library 414, a consistency checker 416, and a user interface simulator 426.

The editor 421 is the application included in the decision support definition editor module 420 that runs on the personal computer or tablet computer that allows a Medical Director to select, edit, and implement decision support tools across a distributed set of medical devices 460. As previously explained, this application can be native to the operating system it is running on, or implemented through a .net/HTML5 implementation. Illustratively, the editor is a native application running on a Windows 8.1 platform. The functionality and operation of the Editor 421 is described in greater detail later.

Directory viewer 422 is an instance of software configured in computing device that interfaces with the decision support definition library 430 to download previous examples of decision support definitions and to save updated versions for use by other Medical Directors. This allows critical thought process and standard of care sharing among the various Medical Directors who operate Emergency Care Systems. These systems might be an Emergency Medical System operating in a pre-hospital environment or an Emergency Room, ICU, Code Team, or Rapid Response Team in a Hospital environment. The directory viewer 422 allows Medical Directors to search for decision support definitions that support specific patient conditions and allows them to view them for applicability with their system.

The decision support definition generator 418 is an application that creates the decision support definition sets that are loaded onto the users allocated medical devices. In other words, the decision support definition generator 418 generates the intelligent agents that are loaded into each of the selected user medical devices. This download is managed through the device management library and supporting tool sets as previously explained that maintain the latest device configuration and software sets for all customer owned devices. As previously explained, software downloads can be delivered to customer's medical devices virtually through network 450, which may be a cloud based communication system. Alternatively, the downloads can be delivered manually through service support tools.

The deployment manager 424 is an application that loads the decision support definition sets into each of the allocated user medical devices. The deployment manager acquires appropriate approvals required before deployment and works in conjunction with the device management library 440 and supporting tool sets to authorize and track implementation on allocated Customer's medical devices. As previously explained, the device management library has the ability to determine how often the decision support definitions are used and what specific user inputs have been captured from each use. The deployment manager can use this data to provide usage metrics to the Medical Director on the various decision support definitions that exist within their system devices.

The template library 412 is a library of components provide a ready-set of common algorithms, containers, functional, and iterators that may be used with the computer system 401 according to this disclosure.

FIG. 4C shows an illustrative template 470 of the template library 412 for use by an Intelligent Agent in the detection of sepsis. According to this model, the existence of sepsis is detected on the presence of “2 or more” of the following rules with definitive evidence of infection:

Temperature >38 degrees C. or <36 degrees C.

Heart Rate >90 beats/min

Respiratory rate >20 respirations/minute

White Blood cell count >12×10̂9/L or <4×10̂9/L or with >10% immature forms

As shown in FIG. 4C, the sepsis threat illustratively has three parts in this example: cues, goal, and course of action. The cue are the rules on individual patient parameters. The rules are shown in FIG. 4C as rules 473, 474, 475, and 476 previously explained. FIG. 4D shows one illustrative template 480 in which the four parameters shown in FIG. 4C may be correlated to each other to express the output rule 472 to have one of the two permissible outputs previously explained. The rule is expressed again in a statement of the rule 482 which states that:
Sepsis=f(2 or more rules A, B, C, D)
   A logical expression 483 of this output rule 471 may illustratively be provided using Boolean logic to express the output rule 910 in the following form:

### If ((Evidence of Infection) && ((A&&B) II (A&&C) II (A&&D) II (B&&C) II (B&&D) II (C&&D)))

For an Intelligent Agent programmed with this rule, logical expression 484 provides an output of logical 1 if true or 0 if not true. If the output is true, the Intelligent Agent has detected sepsis. In the illustrative example, the Intelligent Agent alerts the caregiver by an alert event. For example, the Intelligent Agent may issue a device alert in the form of a display that “Patient meets conditions for Sepsis” as indicated by the following instruction in the protocol.

{System.out.display (“Patient meets conditions for Sepsis”);}

The resource material library 414 is a registry of resources that are available for assisting the Medical Director in the use of the computing system of this disclosure. For example, the resource library may include resources on operation of use, illustrative implementations of computing systems of this disclosure by other Medical Directors, diagnostic information, treatment information, illustrative decision support definitions, etc.

The consistency checker 416 is an application to ensure that all inputs and outputs are fully defined and consistent with the users fielded products. The consistency checker provides consistency checks to allow the user to validate operation prior to implementation.

The user interface simulator 426 is a simulation tool application that allows the Medical Director to simulate how the new decision support definitions will appear on the user interface of the allocated devices. In addition, the user interface simulation tool further allows the Medical Director to visualize how the decision support tool definitions operate on the allocated devices. The user interface information can take the form of messages, checklists, reference material, dosage calculators, whatever types are included in the template library 412 or the resource material library 414. The user interface simulation allows the user to validate operation prior to implementation.

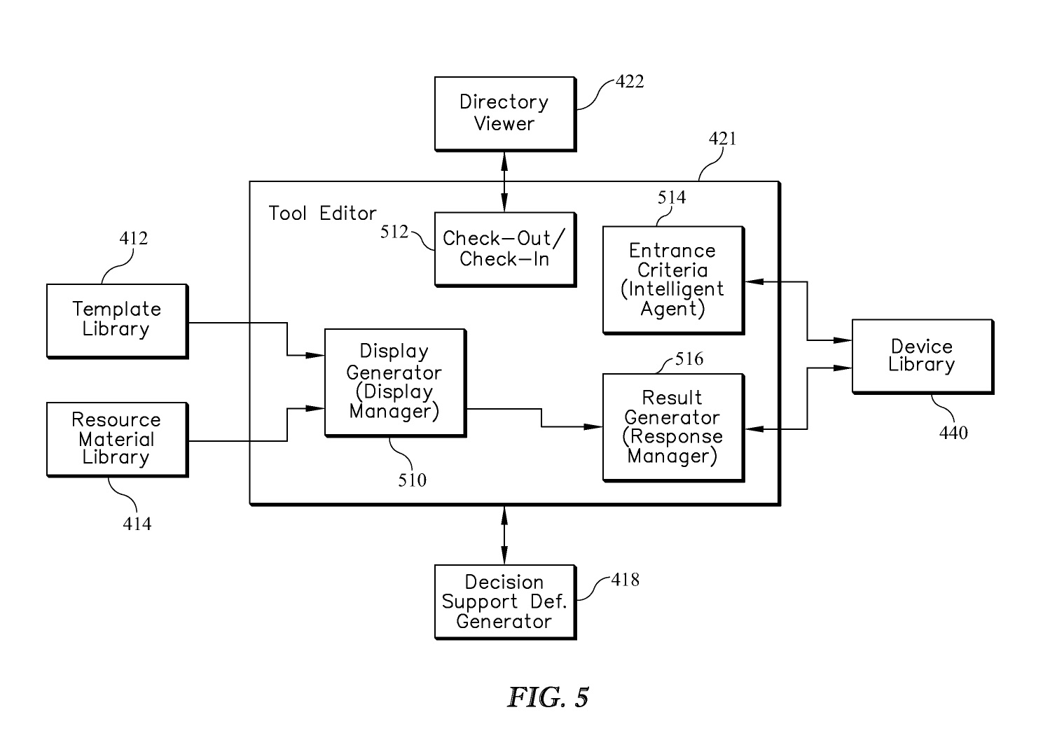

FIG. 5 depicts an illustrative embodiment of the decision support definition editor of the decision support definition editor module shown in FIG. 4B. The decision support definition editor comprises a display generator 510, a check-out/check-in module 512, a result generator 516, and an entrance criteria module 514.

The display generator 510 is an application that defines the specific user interface presentation for a particular decision support definition. The display generator serves as a display manager to manage the information that identifies the respective decision support definition to be displayed on a medical device. For instance, the display generator allows a user to select what data to display on a medical device. The output of the display generator is used by the decision support definition set generator 418 to create the decision support definition sets that are to be loaded into each of the allocated user medical devices via the deployment manager 424. The output of the display generator is applied to the result generator 516 to create the outputs for use by the decision support definition generator 418.

The check-out/check-in module 512 is an application which allows the user to checkout a predefined decision support definition from the directory viewer 422. The Medical Director can assign these tool to specific medical device types or all medical devices in their system. It is possible that a Medical Director may have one version of a decision support definition allocated to their systems Tablet solutions, while a different version is allocated to the systems Team Displays (permanently mounted Wall Display), while a third version is allocated to the systems monitor or monitor with defibrillator. This multiple version approach allows the end user presentation, and resulting outputs to vary, while potentially sharing the same entrance criteria for the decision support definitions. The check-in portion allows the user to publish their custom version of the decision support definition to others via the directory viewer 422.

The result generator 516 is an application that defines what the system will create when any of the user inputs specified by the display generator 510 are selected. The result generator creates the presentation for the data that the display generator has selected to be displayed on a medical device. To do this, the result generator obtains configuration information on the medical devices on which the data is to be presented. The result generator also obtains information that identifies the installation programs that are on the medical devices on which the data is to be presented. Outputs could include visual displays like check marks, shared communications like user inputs and patient events, or display generator transitions like a transition that remove the checklist from the display. The result generator will for example determine what kind of display is possible on a selected medical device based upon the applications available on the selected medical and may scale the to be displayed up or down in order to fit it onto the screen resolution of the particular medical device. Alternatively, if the selected devices do not have the appropriate application to display the selected data, the result generator 516 may obtain the appropriate application from the device library for inclusion into the data package provided for use by the decision support definition generator 418 in generating the decision support definition to create the presentation of data contemplated by the Medical Director for the medical devices. The result generator 516 may allow for shared communications to be sent to other medical devices within the users system which may act as user inputs or trigger stimulus for additional allocated decision support definitions. In this manner, inputs that are provided on one of the medical device types can be detected by the result generator 516 and reflected on other medical device types which may trigger specific behavior by decision support definitions allocated to those medical device types. The result generator communicates with the device management library 440 to determine what possible outputs can be created.

The entrance criteria module 514 is an application that defines the specific input criteria required to trigger a specific decision support definition. It consists of a time based Boolean logic editor that combines available inputs into a Smart Trigger equation. An illustrative Smart Trigger for the detection of sepsis has been previously explained in connection with FIG. 4D. The entrance criteria module communicates to the device library 440 to determine the specific inputs available for this Customer's device type. The outputs of the entrance criteria module are used by the decision support definition set to generate the intelligent agents that are loaded into each of the selected user medical devices.

From the foregoing disclosure it is thus seen that the decision support definitions library 430 stores one or more decision support definitions for providing appropriate treatment paths. The device management library 440 stores device and configuration setting information on one or more devices associated with an organization, the configuration setting including information on smart agents residing on the one or more devices. The decision support definition editor 421 is configured to modify a selected one or more decision support definitions and one or more device and configuration settings. It does so using both the outputs of the entrance criteria module 514 to generate the intelligent agents used in a selected decision support definition that is to be loaded into each of the selected user medical devices and the outputs of the result generator 516 that takes the data from the display generator 510 on (i) selected information that identifies the respective decision support definition to be made available to the medical devices and the (ii) configuration information and (iii) information that identifies an installation program stored in the device library 440 that are available on the medical devices. Using the output from the result generator 516, the decision support definition generator 418 is configured to create the installation configuration file that the deployment manager 424 deploys to the medical devices. The installation configuration file includes an entry for each of a selected one or more decision support definitions, wherein each entry comprises: (i) information that identifies the respective decision support definition, (ii) configuration information, and (iii) information that identifies an installation program. The selected decision support definition illustratively includes any intelligent agent outputs from the entrance criteria module 514. As indicated, the deployment manager is configured to distribute the installation configuration file and the smart agent with the selected decision support definitions to the medical devices.

Once the editing of the support definition is complete, the data sets generated, and the Medical Director has validated the implementation through the provided user interface simulation, the deployment manager 424 is used to acquire appropriate approvals and to work in conjunction with the device management library 440 and supporting tool sets to authorize and track implementation on allocated Customer's medical devices.

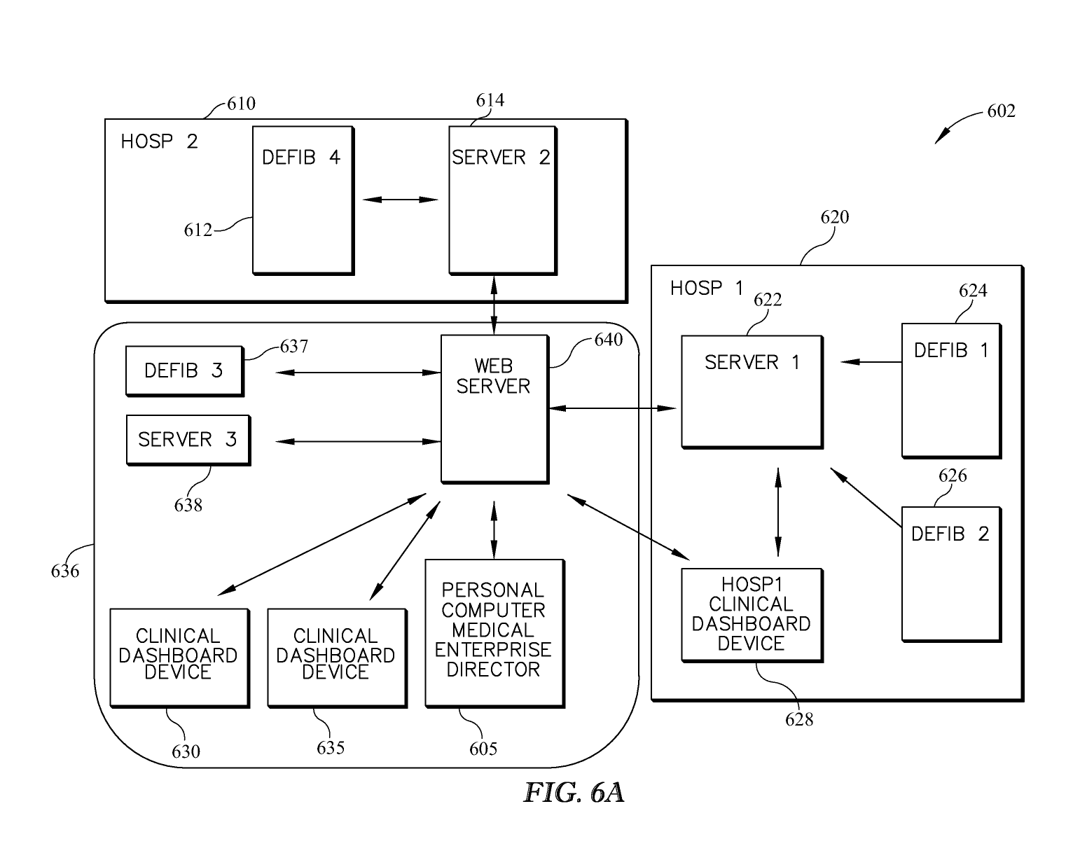

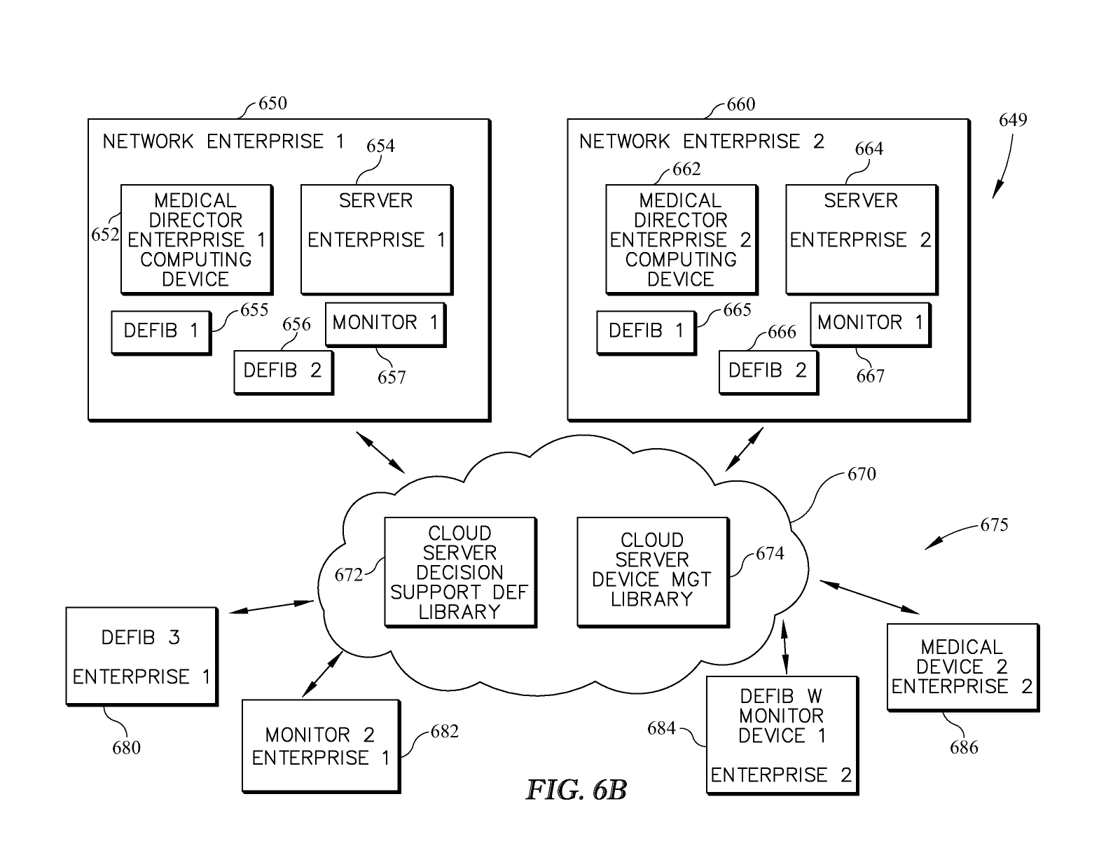

FIG. 6A shows an illustrative functional diagram of an illustrative system illustratively defining a medical enterprise 602 for distributing the installation configuration file smart agent with selected decision support definitions to medical devices according to this disclosure. The system 602 includes hospital networks 610, 620, and a public network 636. Hospital network 610 includes a defibrillator 612 and a server 614 each of which resides in the hospital network 610. Hospital 620 includes a defibrillator 624, a defibrillator 626, a clinical dashboard device 628, and a server 622; each of which also resides in the hospital network 620. Public network includes a web server 640, a defibrillator 637, a clinical dashboard device 630, a clinical dashboard device 635, and a web server 640. The clinical dashboard device 630 and clinical dashboard 636 illustratively reside in a first and a second ambulance that may be associated with either hospital network 610 or hospital network 620 or both. Defibrillator 637 may reside at an office building, an airport, a store, or other public or private place. Server 638 may illustratively be an off-site server of system 401 configured to store and provide the decision support definition library (430 in FIG. 4A) and/or the device management library 440 (element 440 in FIG. 4A) to the system.

The system 603 includes a personal computer 605 of a medical enterprise director used by the medical director to create configuration files and smart agents according to this disclosure. The computer 605 is illustratively shown to reside in the public network such as an office building. Alternatively, the computer 605 may be located anywhere in the system 602 such as in hospital network 610 or hospital network 630 or in any other location in the public network 636.

The medical director configures the configuration files and smart agents on the computer 605 according to this disclosure. The medical director may then distribute the configuration files and smart agents through web server 640 to the defibrillator 637, clinical dashboards 630, 635 in the public network. The configuration files and smart agents may also be distributed through the web server 640 to hospital networks 610 and 620. In the illustrative example, the distribution in each hospital network occurs through a server—server 614 in the case of hospital network 610 and server 622 in the case of hospital 622. These servers distribute the configuration files and smart agents to the medical devices within their hospital network that have been selected for configuration by the current distribution. In the case of hospital network 610, the server 614 distributes the configuration files and smart agents to the defibrillator 612. In the case of hospital 620, the server 622 distributes the configuration files and smart agents to defibrillators 624, 626 and to clinical dashboard device 628. Illustratively, the servers may distribute the configuration files and smart agents to medical devices within the hospital network by local access network lines. Alternatively, the servers may distribute the configuration files and smart agents by wireless transmission such as cellular, WiFi, Rfid, or other wireless or optical transmission. In an alternative embodiment, the distribution of the configuration files and smart agents from computer 605 of the medical director may pass through the web server directly to a medical device in either hospital network 610 or 620 by cellular, WiFi, Rfid, or other wireless or optical transmission.

FIG. 6B shows an alternative illustrative functional diagram of an illustrative system 649 defining a plurality of medical enterprises, for distributing the installation configuration file and the smart agent with selected decision support definitions to medical devices according to this disclosure. System 649 comprises a first network enterprise 650 and a second network enterprise 660. Each of first network enterprise 650 and second network enterprise 660 comprises a computing device of a medical director (computing device 652 in the first network enterprise 650 and computing device 662 in the second network enterprise 660), a server (server 654 in the first network enterprise 650 and server 664 in the second network enterprise), and one or more medical devices (defibrillators 655, 656 and monitor 657 in the first network enterprise 650 and defibrillators 665, 666 and monitor 667 in the second network enterprise). Each of the first network enterprise 650 and the second network enterprise 660 may communicate through cloud 670 with medical devices. For example, network enterprise 650 may communicate with defibrillator 680 and/or monitor 682 in a public network 675 and network enterprise 660 may communicate with defibrillator with monitor device 684 and medical device 686 also in the public network 675. In this illustrative embodiment, system 649 further comprises a cloud server decision support definition library 672 and a cloud server device management library 674.

Both libraries 672, 674 illustratively reside in the cloud. Advantageously, the cloud server decision support definition library 672 provides a Web Based store to share potential decision support definitions with other Customers. For instance each of network enterprises 650, 660 may upload and download decision support definitions into the cloud server decision support definition library 672 Web Based store; each thereby making the decision support definitions they create or use available to the other. In another example each of network enterprises 650, 660 may upload and download decision device management data into the cloud server device management library 674 Web Based store; each thereby making the decision support definitions they create or use available to the other. Each Web Based store may illustratively be on a subscription basis where access to the store is by paid subscription. Alternatively, access may be granted based on other basis such as by permissions agreed upon by participating members.

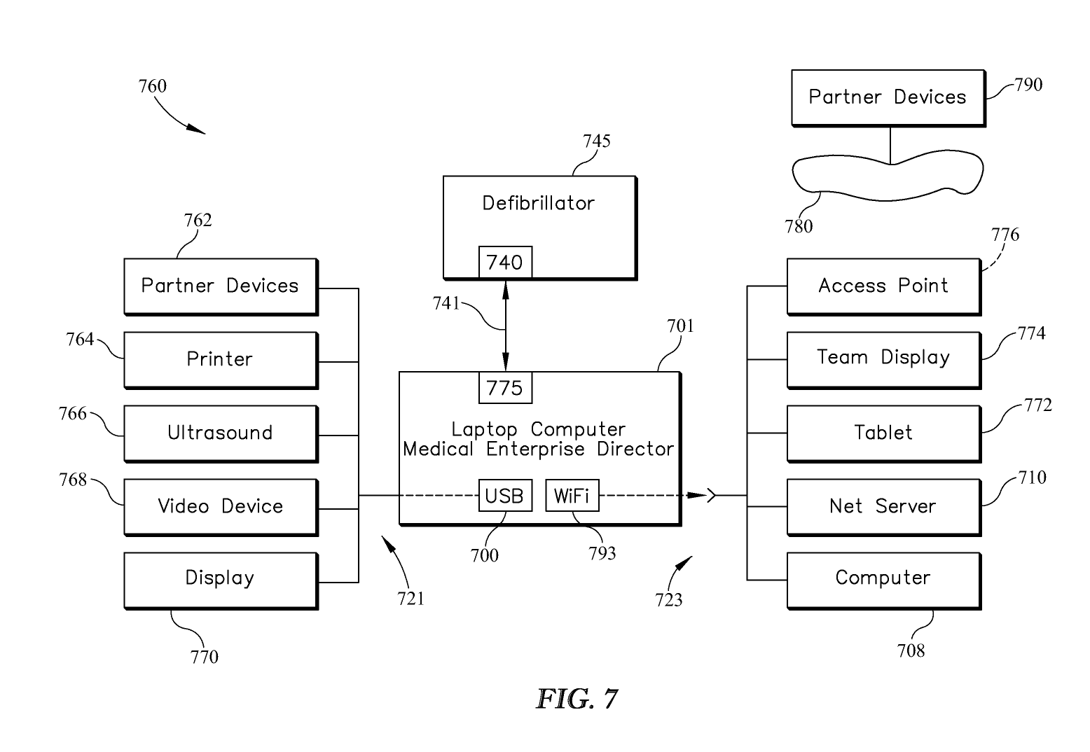

FIG. 7 shows an illustrative functional diagram of an alternative illustrative system 760, illustratively defining a medical enterprise, for distributing the installation configuration file and the smart agent with selected decision support definitions to medical devices according to this disclosure. The system 760 defining the medical enterprise shown in FIG. 7 comprises a laptop computer 701 of a medical enterprise director which is tethered by a USB connector 700 and a wired connection 721 to one or more of a display 770, a video device 768, an ultrasound device 766, a printer 764, and partner devices 762. Laptop computer 701 may also be connected directly to a local defibrillator 745, for example by a wired connection 741 through a port 740 of the defibrillator and a port 775 of the laptop computer. The laptop computer 701 of the medical enterprise director is also wirelessly connected 793 through a local area network 723 to a computer 708, a net server 710, a tablet 772, a team display 774, and an access point 776. The access point 776 allows the laptop computer 701 of the medical enterprise director to communicate over the Internet 780 with a remote utility 790. Remote utility 790 may be a utility such as a defibrillator or a medical device that is being used in the public network. Remote utility may also be a server for a remote local area network of the medical enterprise. In this instance, the server may be serving defibrillators and other medical devices operating inside the remote local area network. The remote utility may also be a server that is shared between a plurality of medical enterprises or other entities. For instances, two or more medical enterprises may pool their installation configuration files and the smart agents in order to provide for a more robust set of installation configuration files and the smart agents to be available to their respective medical enterprises. As another example, an individual professional, such as a doctor, an entity of professionals, such as a medical clinic, or an entity of professionals trained in defining installation configuration files and smart agents may also be given access to the server that is represented by the remote utility 790 in this example.

It will be appreciated from the description accompanying FIGS. 6 and 7 that a computing device of the medical enterprise director may be used to distribute installation configuration files and the smart agents with selected decision support definitions according to this disclosure to medical devices throughout any private, public, and private and public enterprises that make up the medical enterprise overseen by the medical director. While the computing devices used by the medical enterprise director are illustratively shown as a personal computer and a laptop computer in FIGS. 6 and 7, respectively, it will be appreciated that any computing device powerful enough to execute the instructions of the decision support definition editor of this disclosure may be used. Illustratively, the computing device may also be configured to execute the decision support definition generator and the deployment manager. Alternatively, the decision support definition generator and/or the deployment manager may be executed by another computing device, such as a web server, in which case the computing device of the medical enterprise director may communicate with the server to execute these functionalities. Illustratively, the device management library and the decision support definitions library may be executed by another computing device, such as a web server, in which case the computing device of the medical enterprise director may communicate with the server to execute these functionalities. Alternatively, all of the foregoing functionalities may be executed on the computing device of the medical enterprise director.

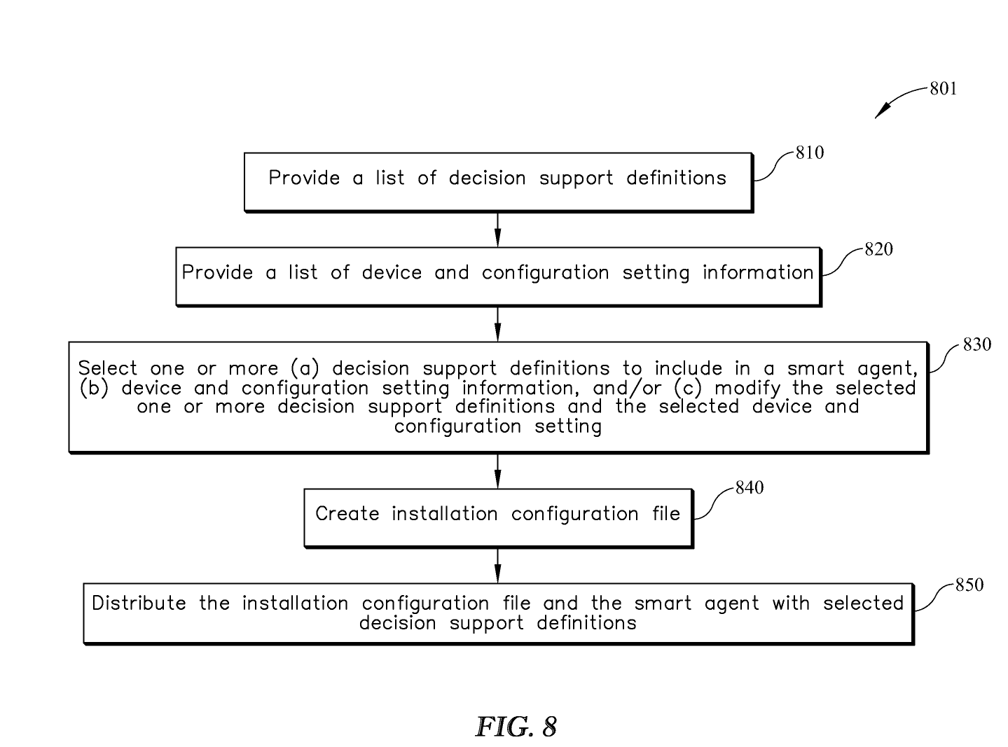

FIG. 8 depicts an illustrative embodiment of a computer-implemented method 801 for providing a configurable frame work for creation and distribution of smart agents according to this disclosure.

In step 810, a list of decision support definitions for providing appropriate treatment paths is provided. In step 820, a list of device and configuration setting information on one or more devices associated with an organization is provided. Illustratively, the configuration setting includes information on smart agents residing on the one or more devices. In step 830, the medical enterprise director selects (a) one or more decision support definitions from the list of decision support definitions to include in a smart agent, (b) one or more device and configuration settings on the one or more devices associated with an organization, and/or (c) modifying the selected one or more decision support definitions and the selected one or more device and configuration settings. In step 840, an installation configuration file is created comprising an entry for each of a selected one or more decision support definitions taken from the decision support definitions library. Illustratively, each entry for the selected one or more decision support definitions comprises: (i) information that identifies the respective decision support definition, (ii) configuration information on one or more devices associated with an organization taken from the device management library to be provided the installation configuration file, and (iii) information that identifies an installation program to use in installing the respective decision support definitions on one or more devices associated with an organization to be provided the installation configuration file. In step 850, the installation configuration file and the smart agent with selected decision support definitions is distributed to one or more medical devices in the medical enterprise.

In an alternative embodiment, the method further comprises the step of distributing one or more installation programs to use to install the respective decision support definitions on the one or more devices associated with an organization to be provided the installation configuration file and the smart agent. In yet another embodiment, the method further includes a step of securing appropriate approval prior to distribution of the configuration file and the smart agent.

In an alternative embodiment, the method may further comprise the steps of: determining data on how often the decision support definitions are used; and using this data to provide usage metrics. In another embodiment, the method of further comprise the steps of: uploading the modified selected one or more decision support definitions and/or the modified one or more device and configuration settings from a decision support editor to a decision support definition library.

One of the features of this disclosure is that the medical enterprise director may simulate the operation of the smart agent at any time. In this embodiment, the method the method further comprises the step of: simulating operation of the smart agent on the one or more devices associated with an organization to be provided the installation configuration file and the smart agent.

The method of this disclosure may enable the medical enterprise director with templates and resources for use in modifying the selected one or more decision support definitions and the selected one or more device and configuration settings. In this embodiment, the method may further include the step of: providing a library of one or more template including one or more standards for the modification of decision support definitions for inclusion in the smart agent and/or providing a library of resource materials including one or more sources of information for use in the modification of decision support definitions for inclusion in the smart agent.

It is important that the medical enterprise director ensure that all inputs and outputs to and from a decision support definition the installation configuration file and the smart agent are consistent with the decision support definitions and installation configuration files either implemented on a medical device or supportable by a medical device to be distributed the decision support definitions and installation configuration files. To enable this feature, the method of this disclosure may further comprise the step of performing a consistency check to ensure that all inputs and outputs to and from a decision support definition the installation configuration file and the smart agent.

It is especially important that the decision support definitions are properly presented for rendering on a display of a medical device to be distributed the decision support definitions. To enable this feature, the disclosed process may include a step of defining a specific user interface presentation for the one or more standards from the library of templates for the modification of decision support definitions for inclusion in the smart agent. Alternatively, or in addition, the method may further include the step of defining a specific user interface presentation for the one or more sources of information from the library of resource material for use in the modification of decision support definitions for inclusion in the smart agent on a display. To ensure that the medical device is provided with the proper configuration settings for rendering the presentation, the method may further include the step of including the specific user interface presentation in the installation configuration file.

To provide a rich user interface, the method of this disclosure may further include the step of providing a visual tool through which the user can select fields of data for the method to generate a data retrieval procedure for the smart agent based on the fields selected by the user. The fields of data included in the visual tool may be selected from the group consisting of messages, checklists, reference material, dosage calculators, and information included in standards included in the template library or in resource materials included in the resource material library.

The method may define specific input data criteria required for triggering a decision support definition. In this event, the method may further include the step of defining specific input data criteria required to trigger a decision support definition selected by the user. The method may utilize a time based Boolean logic editor to define specific input data criteria that combines available data inputs into a smart trigger equation for use by the smart agent. The specific data input criteria required to trigger a decision support definition may be based upon data inputs available to the one or more devices associated with an organization to be provided the installation configuration file and the smart agent.

The method may further comprise the step of distributing the installation configuration file and the smart agent with selected decision support definitions over the internet. The method may be implemented on an operating environment selected from the group consisting of personal computers, server computers, handheld or laptop devices, multi-processor systems, microprocessor-based systems, programmable consumer electronics, network personal computers, minicomputers, mainframe computers, and distributed computing environments that include any of the above systems or devices.

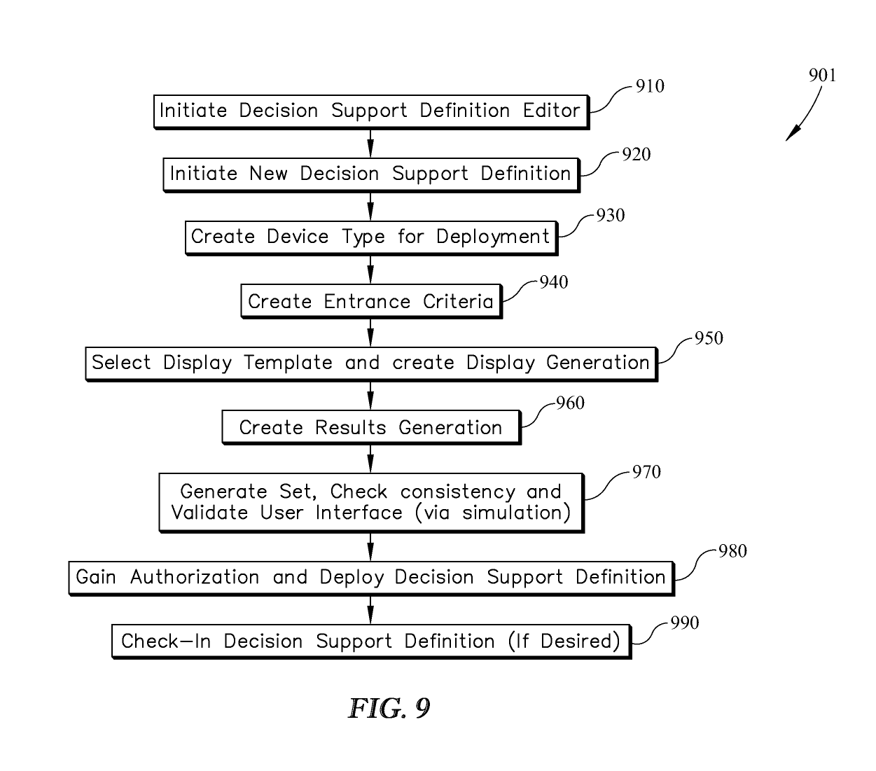

FIG. 9 depicts an illustrative process 901 for selecting, editing, and deploying a new smart agent according to this disclosure. In step 910, a medical enterprise director initiates the decision support definition editor. In step 920, the director initiates a new decision support definition. In step 930, the director selects a device type for deployment. In step 940, the director creates the entrance criteria. In step 950, the director selects the display template and creates the display generation. In step 960, the director creates the results generation. In step 970, the director generates the set of decision support definitions, checks the consistency of the decision support definitions, and validates the user interface via a simulation. In step 980, the director gains authorization to deploy and deploys the generated set of decision support definitions. In step 990, the director checks-in, that is to say, uploads the generated set of decision support definitions into the decision support definition library (element 430 in FIG. 4) if desired.

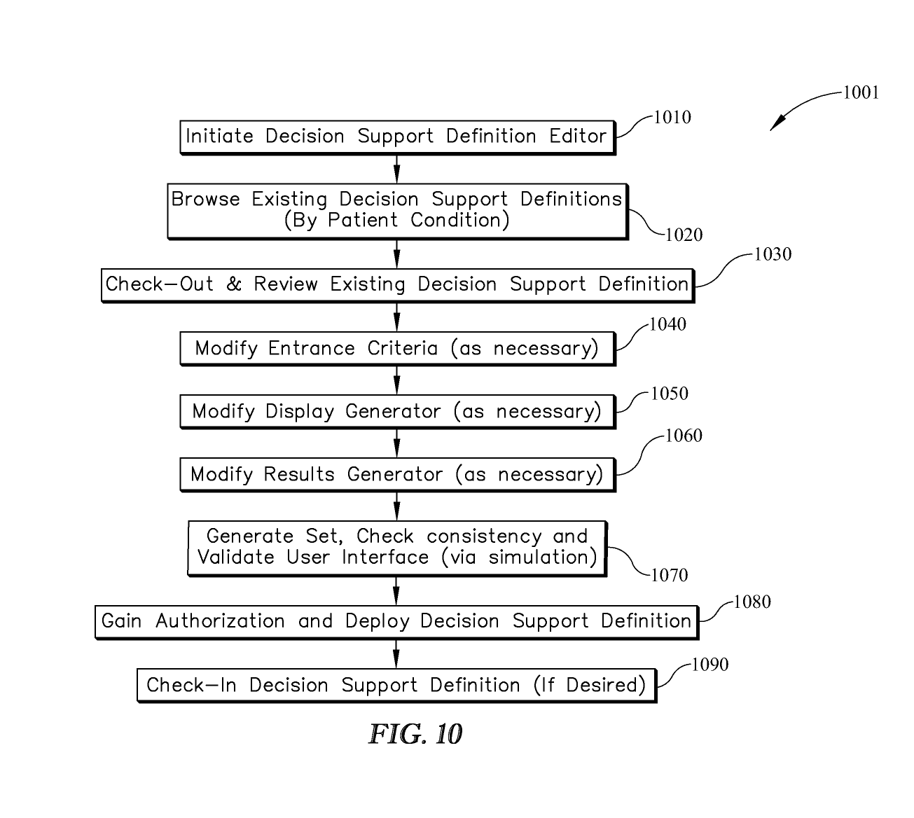

FIG. 10 depicts an illustrative process 1001 for selecting, editing, and deploying an existing smart agent according to this disclosure. In step 1010, a medical enterprise director initiates the decision support definition editor. In step 1020, the director browses existing decision support definitions by patient condition. In step 1030, the director checks-out, that is to say, downloads the existing decision support definition into the editor and reviews the definition. In step 1050, the director modifies the entrance criteria as necessary. In step 1050, the director modifies the display generation as necessary. In step 1060, the director modifies the results generation as necessary. In step 1070, the director generates the set of decision support definitions, checks the consistency of the decision support definitions, and validates the user interface via a simulation. In step 1080, the director gains authorization to deploy and deploys the generated set of decision support definitions. In step 1090, the director checks-in, that is to say, uploads the generated set of decision support definitions into the decision support definition library (element 430 in FIG. 4) if desired.

There is thus disclosed a computer method and system that provides a configurable frame work for creation and distribution of smart agents. A decision support definitions library stores one or more decision support definitions for providing appropriate treatment paths. A device management library stores device and configuration setting information on one or more devices associated with an organization, the configuration setting including information on smart agents residing on the one or more devices. A decision support definition editor is configured to modify a selected one or more decision support definitions and one or more device and configuration settings. A decision support definition generator is configured to create an installation configuration file including an entry for each of a selected one or more decision support definitions, wherein each entry comprises: (i) information that identifies the respective decision support definition, (ii) configuration information, and (iii) information that identifies an installation program. A deployment manager is configured to distribute the installation configuration file and the smart agent with selected decision support definitions.

The deployment manager may be further configured to distribute one or more installation programs to use to install the respective decision support definitions on the one or more devices associated with an organization to be provided the installation configuration file and the smart agent. The deployment manager may be further configured to require appropriate approval prior to distribution of the configuration file and the smart agent.

The device management library may be further configured to determine how often the decision support definitions are used and deployment manager is further configured to use this data to provide usage metrics.

The system may be provided with a directory viewer configured to download decision support definitions from the decision support definition library to the decision support editor and to upload decision support definitions modified by the decision support definition editor from the decision support editor to the decision support definition library. The system may include a user interface simulator configured to simulate operation of the smart agent on the one or more devices associated with an organization to be provided the installation configuration file and the smart agent. The system may include a template library including one or more standards for the modification of decision support definitions for inclusion in the smart agent. The system may include a resource material library including one or more sources of information for use in the modification of decision support definitions for inclusion in the smart agent.

The system may include a consistency checker configured to ensure that all inputs and outputs to and from the decision support definition generator are fully defined and consistent with the one or more devices associated with an organization to be provided the installation configuration file and the smart agent.

The decision support definition editor may further comprises a check-in/out portion; the check-in portion being configured to download the decision support definitions from the decision support definition library to the decision support editor and the check-out portion being configured to upload the decision support definitions modified by the decision support definition editor from the decision support editor to the decision support definition library. The decision support definition editor may further include a display generator for defining a specific user interface presentation for the one or more standards from the template library for the modification of decision support definitions for inclusion in the smart agent. The decision support definition may further include a display generator for defining a specific user interface presentation for the one or more sources of information from the resource material library for use in the modification of decision support definitions for inclusion in the smart agent on a display. An output of the display generator is applied to the decision support definition generator for inclusion in the installation configuration file.

The decision support definition editor may further include a result generator configured to provide a visual tool through which the user can select fields of data for the system to generate a data retrieval procedure for the smart agent based on the fields selected by the user. The fields of data is selected from the group consisting of messages, checklists, reference material, dosage calculators, and information included in standards included in the template library or in resource materials included in the resource material library.

The decision support definition editor may further include an entrance criteria element configured to define specific input data criteria required to trigger a decision support definition selected by the use. The entrance criteria element includes a time based Boolean logic editor that combines available data inputs into a smart trigger equation for use by the smart agent. The specific data input criteria required to trigger a decision support definition may be based upon data inputs available to the one or more devices associated with an organization to be provided the installation configuration file and the smart agent. An output of the entrance criteria element may be applied to the decision support definition generator to generate the smart agent.

The installation configuration file and the smart agent with selected decision support definitions may distributed by the deployment manager over the internet. The system may be implemented on an operating environment selected from the group consisting of personal computers, server computers, handheld or laptop devices, multi-processor systems, microprocessor-based systems, programmable consumer electronics, network personal computers, minicomputers, mainframe computers, and distributed computing environments that include any of the above systems or devices.

A computer-implemented method for provides a configurable frame work for creation and distribution of smart agents may include providing a list of decision support definitions for providing appropriate treatment paths. A list of device and configuration setting information may be provided on one or more devices associated with an organization, the configuration setting including information on smart agents residing on the one or more devices. There may be a selection of (a) one or more decision support definitions from the list of decision support definitions to include in a smart agent, (b) one or more device and configuration settings on the one or more devices associated with an organization, and/or (c) modifying the selected one or more decision support definitions and the selected one or more device and configuration settings. An installation configuration file may be created including an entry for each of a selected one or more decision support definitions taken from the decision support definitions library, wherein each entry for the selected one or more decision support definitions comprises: (i) information that identifies the respective decision support definition, (ii) configuration information on one or more devices associated with an organization taken from the device management library to be provided the installation configuration file, and (iii) information that identifies an installation program to use in installing the respective decision support definitions on one or more devices associated with an organization to be provided the installation configuration file. The installation configuration file and the smart agent with selected decision support definitions may be distributed.

One or more installation programs may be distributed to use to install the respective decision support definitions on the one or more devices associated with an organization to be provided the installation configuration file and the smart agent. The appropriate approval may be secured prior to distribution of the configuration file and the smart agent. Data on how often the decision support definitions are used may be determined; and this data may be used to provide usage metrics. The modified selected one or more decision support definitions and/or the modified one or more device and configuration settings may be uploaded from a decision support editor to a decision support definition library. The decision support definition library or the device management library or both reside on a cloud server.

The operation of the smart agent may be simulated on the one or more devices associated with an organization to be provided the installation configuration file and the smart agent. A library may be provided of one or more template including one or more standards for the modification of decision support definitions for inclusion in the smart agent. A library of resource materials may be provided including one or more sources of information for use in the modification of decision support definitions for inclusion in the smart agent. A consistency check may be performed to ensure that all inputs and outputs to and from a decision support definition the installation configuration file and the smart agent. A specific user interface presentation may be defined for the one or more standards from the library of templates for the modification of decision support definitions for inclusion in the smart agent. A specific user interface presentation may be defined for the one or more sources of information from the library of resource material for use in the modification of decision support definitions for inclusion in the smart agent on a display.

The specific user interface presentation may be included in the installation configuration file. A visual tool may be provided through which the user can select fields of data for the method to generate a data retrieval procedure for the smart agent based on the fields selected by the user. The fields of data may be selected from the group consisting of messages, checklists, reference material, dosage calculators, and information included in standards included in the template library or in resource materials included in the resource material library. The specific input data criteria required to trigger a decision support definition selected by the user may be defined. The entrance criteria element may include a time based Boolean logic editor that combines available data inputs into a smart trigger equation for use by the smart agent. The specific data input criteria required to trigger a decision support definition may be based upon data inputs available to the one or more devices associated with an organization to be provided the installation configuration file and the smart agent.

The installation configuration file and the smart agent with selected decision support definitions may be distributed over the internet. The method may be implemented on an operating environment selected from the group consisting of personal computers, server computers, handheld or laptop devices, multi-processor systems, microprocessor-based systems, programmable consumer electronics, network personal computers, minicomputers, mainframe computers, and distributed computing environments that include any of the above systems or devices. The decision support definition library or the device management library or both reside on a cloud server.

The system and method illustratively include an application that may run on any personal or tablet computer that has enterprise network access or public Internet access. The application allows the Medical Director to download existing decision support definitions, edit them, create new versions, allocate them to their specific medical devices, and share them with others. It provides error checking to make sure the new decision support definitions are compatible with the Medical Director's system. The editor may come with version control and approval authority, so the Medical Director can actively manage what decision support definitions are currently available on their medical device types and works in conjunction with a device management tool to set to load the new decision support definition sets onto the various allocated medical devices. A Medical Director can track deployment/usage metrics helping to keep track of what devices have what tools, how the tools are being used, and if compliance to the tool is desired, how often the tool is being successfully used by the end user.

The disclosed system and method provides a flexible structure for a decision support definition editor with respect to the scope of patient care paths that can be supported, the approach to managing device configuration and allocation of decision support definitions. The disclosed system and method also provides for a Web Based store to share potential decision support definitions with other Customers.

The disclosure provides a decision support definition editor that allows Medical Directors to create decision support definitions that operate on their medical devices. The decision support definitions may be used for an emergency medical response system to aid in the diagnosis and treatment of emergent patients. The decision support definition editor may be customizable (tailored) by the customer to support any patient condition using any standard of care the customer chooses. The decision support definition editor may reflect the customers own diagnostic algorithms, care paths and terminology. The decision support definition editor may be deployed to one or all device types within a customer's emergency medical response system. The decision support definition editor may be customize/tailored to appear differently on the various devices. The decision support definition editor may present the decision support definition in many forms to the Clinical User such as messaging, checklists, reference material, dosage calculators, etc.). The decision support definition editor may allow the Customer to specify how many and what type of user interface controls appear on the screen. The decision support definition editor may allow the Customer to select what the response to any User input will be in the system. The decision support definition editor may expand in User Interface capability over time. The decision support definition editor may operate as a thick client or thin client solutions. The decision support definition editor may provide a decision support definition with decision support library access for sharing tools among Customers. The decision support definition editor supports authorization and deployment of the decision support definitions via an integrated device management system. The decision support definition editor may be deployed through a device management library and supporting tool sets. The decision support definition editor may provide metrics on usage and user compliance (proper response to messages and checklist items). The decision support definition editor may supports various business models for charging the Customer (e.g., by use, by tool implemented, by devices deployments, etc.)

In this description, numerous details have been set forth in order to provide a thorough understanding. In other instances, well-known features have not been described in detail in order to not obscure unnecessarily the description.

A person skilled in the art will be able to practice the present disclosure in view of this description, which is to be taken as a whole. The specific embodiments as disclosed and illustrated herein are not to be considered in a limiting sense. Indeed, it should be readily apparent to those skilled in the art that what is described herein may be modified in numerous ways. Such ways can include equivalents to what is described herein. In addition, the disclosure may be practiced in combination with other systems. The following claims define certain combinations and sub-combinations of elements, features, steps, and/or functions, which are regarded as novel and non-obvious. Additional claims for other combinations and sub-combinations may be presented in this or a related document.

1. A computer system that provides a configurable frame work for creation and distribution of smart agents, the system comprising:
a processor and memory configured to execute software instructions;
a decision support definitions library configured to store one or more decision support definitions for providing appropriate treatment paths;
a device management library configured to store one or more device and configuration settings on one or more devices associated with an organization, the configuration settings including information on smart agents residing on the one or more devices;
a decision support definition editor configured to:
(a) select one or more of the decision support definitions in the list of decision support definitions to include in a smart agent,
(b) select one or more of the device and configuration settings on the one or more devices associated with an organization, and
(c) one or both of:
(i) modify the selected one or more decision support definitions and
(ii) modify the selected one or more device and configuration settings;
a decision support definition generator configured to create an installation configuration file comprising an entry for each of the selected one or more decision support definitions taken from the decision support definitions library, wherein each entry for the selected one or more decision support definitions comprises:
(i) information that identifies the respective decision support definition,
(ii) configuration information on one or more devices associated with an organization taken from the device management library to be provided the installation configuration file, and
(iii) information that identifies an installation program to use in installing the respective decision support definitions on one or more devices associated with the organization to be provided the installation configuration file; and
a deployment manager configured to distribute the installation configuration file and the smart agent with the selected decision support definitions.

2. The system of claim 1 wherein the deployment manager is further configured to distribute one or more installation programs to use to install the respective decision support definitions on the one or more devices associated with an organization to be provided the installation configuration file and the smart agent.

3. The system of claim 1 wherein the deployment manager is further configured to require approval prior to distribution of the configuration file and the smart agent.

4. The system of claim 1 wherein the device management library is further configured to determine the frequency with which the decision support definitions are used and the deployment manager is further configured to use the determined frequency to calculate usage metrics.

5. The system of claim 1 further comprising a directory viewer configured to download decision support definitions from the decision support definition library to the decision support editor and to upload decision support definitions modified by the decision support definition editor from the decision support editor to the decision support definition library.

6. The system of claim 1 further comprising a user interface simulator configured to simulate operation of the smart agent on the one or more devices associated with an organization to be provided the installation configuration file and the smart agent.

7. The system of claim 1 further comprising a template library including one or more standards for the modification of decision support definitions for inclusion in the smart agent.

8. The system of claim 1 further comprising a resource material library including one or more sources of information for use in the modification of decision support definitions for inclusion in the smart agent.

9. The system of claim 1 further comprising a consistency checker configured to ensure that all inputs and outputs to and from the decision support definition generator are fully defined and consistent with the one or more devices associated with an organization to be provided the installation configuration file and the smart agent.

10. The system of claim 1, the decision support definition editor further comprising a check-in portion and a check-out portion, the check-in portion being configured to download the decision support definitions from the decision support definition library to the decision support editor and the check-out portion being configured to upload the decision support definitions modified by the decision support definition editor from the decision support editor to the decision support definition library.

11. The system of claim 7, the decision support definition editor further comprising a display generator for defining a specific user interface presentation for the one or more standards from the template library for the modification of decision support definitions for inclusion in the smart agent.

12. The system of claim 8, the decision support definition editor further comprising a display generator for defining a specific user interface presentation for the one or more sources of information from the resource material library for use in the modification of decision support definitions for inclusion in the smart agent on a display.

13. The system of claim 11 wherein an output of the display generator is applied to the decision support definition generator for inclusion in the installation configuration file.

14. The system of claim 1, the decision support definition editor further comprising a result generator configured to provide a visual tool through which the user selects fields of data for the system to generate a data retrieval procedure for the smart agent based on the fields selected by the user.

15. The system of claim 14 wherein the fields of data are selected from the group consisting of:
messages,
checklists,
reference material,
dosage calculators, and
information included in:
standards included in a template library for the modification of decision support definitions for inclusion in the smart agent, or
resource materials included in a resource material library for use in the modification of decision support definitions for inclusion in the smart agent.

16. The system of claim 1, the decision support definition editor further comprising an entrance criteria element configured to define specific input data criteria required to trigger a decision support definition selected by the user.

17. The system of claim 16, the entrance criteria element further comprising a time based Boolean logic editor that combines available data inputs into a smart trigger equation for use by the smart agent.

18. The system of claim 16 wherein the specific data input criteria required to trigger a decision support definition are based upon data inputs available to the one or more devices associated with an organization to be provided the installation configuration file and the smart agent.

19. The system of claim 16 wherein an output of the entrance criteria element is applied to the decision support definition generator to generate the smart agent.

20. The system of claim 1 wherein the installation configuration file and the smart agent with selected decision support definitions are distributed by the deployment manager over the internet.

21. The system of claim 1 wherein the system is implemented on an operating environment including one or more of personal computers, server computers, handheld or laptop devices, multi-processor systems, microprocessor-based systems, programmable consumer electronics, network personal computers, minicomputers, mainframe computers, and distributed computing environments that include any of the above systems or devices.

22. The system of claim 1 wherein either the decision support definition library or the device management library or both reside on a cloud server.

23. A computer-implemented method for providing a configurable frame work for creation and distribution of smart agents, the method comprising:
providing a list of decision support definitions for providing appropriate treatment paths;
providing a list of device and configuration settings on one or more devices associated with an organization, the configuration settings including information on smart agents residing on the one or more devices;
selecting:
(a) one or more decision support definitions from the list of decision support definitions to include in a smart agent,
(b) one or more device and configuration settings on the one or more devices associated with an organization, and
(c) one or both of:
(i) modifying the selected one or more decision support definitions and
(ii) modifying the selected one or more device and configuration settings;
creating an installation configuration file comprising an entry for each of the selected one or more decision support definitions taken from the decision support definitions library, wherein each entry for the selected one or more decision support definitions comprises:
(i) information that identifies the respective decision support definition,
(ii) configuration information on one or more devices associated with an organization taken from the device management library to be provided the installation configuration file, and
(iii) information that identifies an installation program to use in installing the respective decision support definitions on one or more devices associated with an organization to be provided the installation configuration file; and
distributing the installation configuration file and the smart agent with selected decision support definitions.

24. The method of claim 23 further comprising the step of:
distributing one or more installation programs to use to install the respective decision support definitions on the one or more devices associated with an organization to be provided the installation configuration file and the smart agent.

25. The method of claim 23 further comprising the step of:
securing approval prior to distribution of the configuration file and the smart agent.

26. The method of claim 23 further comprising the steps of:
determining frequency data on how often the decision support definitions are used; and using the frequency data to calculate usage metrics.

27. The method of claim 23 further comprising the steps of:
uploading one or both of the modified selected one or more decision support definitions and the modified one or more device and configuration settings from a decision support editor to a decision support definition library.

28. The method of claim 23 further comprising the step of:
simulating operation of the smart agent on the one or more devices associated with an organization to be provided the installation configuration file and the smart agent.

29. The method of claim 23 further comprising the step of:
providing a library of one or more templates including one or more standards for the modification of decision support definitions for inclusion in the smart agent.

30. The method of claim 23 further comprising the step of:
providing a library of resource materials including one or more sources of information for use in the modification of decision support definitions for inclusion in the smart agent.

31. The method of claim 23 further comprising the step of:
performing a consistency check to ensure that all inputs and outputs to and from a decision support definition include the installation configuration file and the smart agent.

32. The method of claim 29 further comprising the step of:
defining a specific user interface presentation for the one or more standards from the library of templates for the modification of decision support definitions for inclusion in the smart agent.

33. The method of claim 30 further comprising the step of:
defining a specific user interface presentation for the one or more sources of information from the library of resource material for use in the modification of decision support definitions for inclusion in the smart agent on a display.

34. The method of claim 32 further comprising the step of:
including the specific user interface presentation in the installation configuration file.

35. The method of claim 23 further comprising the step of:
providing a visual tool through which the user selects fields of data for the method to generate a data retrieval procedure for the smart agent based on the fields selected by the user.

36. The method of claim 35 wherein the fields of data are selected from the group consisting of:
messages,
checklists,
reference material,
dosage calculators, and
information included in:
standards included in a template library for the modification of decision support definitions for inclusion in the smart agent, or
resource materials included in a resource material library for use in the modification of decision support definitions for inclusion in the smart agent

37. The method of claim 23 further comprising the step of:
defining an entrance criteria element configured to define specific input data criteria required to trigger a decision support definition selected by the user.

38. The method of claim 37 wherein the entrance criteria element includes a time based Boolean logic editor that combines available data inputs into a smart trigger equation.

39. The method of claim 37 wherein the specific data input criteria required to trigger a decision support definition are based upon data inputs available to the one or more devices associated with an organization to be provided the installation configuration file.

40. The method of claim 39 wherein the defined specific input data criteria required to trigger a decision support definition selected by the user is used to generate a smart agent.

41. The method of claim 23 further comprising the step of:
distributing the installation configuration file and the smart agent with selected decision support definitions over the internet.

42. The method of claim 23 wherein the method is implemented on an operating environment that includes one or more of personal computers, server computers, handheld or laptop devices, multi-processor systems, microprocessor-based systems, programmable consumer electronics, network personal computers, minicomputers, mainframe computers, and distributed computing environments that include any of the above systems or devices.

43. The method of claim 23 wherein the provided list of decision support definitions for providing appropriate treatment paths, the provided list of device and configuration setting information on one or more devices associated with an organization, or both reside on a cloud server.
